(19) United States
(12) Patent Application Publication
(10) Pub. No.: US 2011/0186434 A1
(43) Pub. Date: Aug. 4, 2011

(54) QUALITATIVE AND/OR QUANTITATIVE DETERMINATION OF A PROTEINACEOUS MOLECULE IN A PLURALITY OF SAMPLES

(75) Inventors: Kai Naumann, Halle (DE); Dierk Scheel, Halle (DE)

(73) Assignee: Leibniz Institute of Plant Biochemistry

(21) Appl. No.: 13/055,384

(22) PCT Filed: Jul. 23, 2009
(86) PCT No.: PCT/EP09/59513

§ 371 Date: Apr. 19, 2011

(30) Foreign Application Priority Data
Jul. 23, 2008 (EP) 08160997.6

(51) Int. Cl. G01N 27/447; G01N 33/53

(52) U.S. Cl. 204/549; 204/645

(57) ABSTRACT

The present invention relates to the qualitative and/or quantitative determination of a proteinaceous molecule in a plurality of samples by combining a particular labeling and fractionation strategy. In particular, each sample is provided with a dye chosen from a set of dyes, wherein each dye within the set of dyes emits luminescent light at a wavelength that is sufficiently different from the emitted luminescent light of the remaining dyes in the set of dyes to provide a different light signal when excited. Subsequent to labelling the proteinaceous molecule with the fluorescent dye, the samples are combined and fractionated by means of electrophoresis, the labeled proteinaceous molecule having a relative electrophoretic mobility that differs from the electrophoretic mobility of the proteinaceous molecule labeled with another dye within the set of dyes. After capturing separate images of the labeled proteinaceous molecule at different wavelengths, the images are aligned by means of image processing. The present invention further relates to a kit and a set of luminescent dyes for use in qualitatively and/or quantitatively determining a proteinaceous molecule in a plurality of samples by means of electrophoresis.

### SUBJECT OF THE INVENTION

The present invention relates to the qualitative and/or quantitative determination of a proteinaceous molecule in a plurality of samples by combining a particular labeling and fractionation strategy. In particular, each sample is provided with a dye chosen from a set of dyes, wherein each dye within the set of dyes emits luminescent light at a wavelength that is sufficiently different from the emitted luminescent light of the remaining dyes in the set of dyes to provide a different light signal when excited. Subsequent to labelling the proteinaceous molecule with the fluorescent dye, the samples are combined and fractionated by means of electrophoresis, the labeled proteinaceous molecule having a relative electrophoretic mobility that differs from the electrophoretic mobility of the proteinaceous molecule labeled with another dye within the set of dyes. After capturing separate images of the labeled proteinaceous molecule at different wavelengths, the images are aligned by means of image processing. The present invention further relates to a kit and a set of luminescent dyes for use in qualitatively and/or quantitatively determining a proteinaceous molecule in a plurality of samples by means of electrophoresis.

### BACKGROUND OF THE INVENTION

One of the most widely used methods for the separation of proteins is gel electrophoresis. A protein sample on an inert support is subjected to an electric field that causes the proteins to migrate according to molecular weight. Typically, the supports are made of polymers, such as polyacrylamide, which is a copolymer of acrylamide and bisacrylamide, or agarose, a polymer of glucose units. In the most common method of electrophoretic separation of proteins, typically abbreviated SDS-PAGE, proteins are coated with sodium dodecyl sulfate (SDS) and placed in a support of polyacrylamide gel and subjected to an electric field. However, the separated proteins are not generally visible to the naked eye. Therefore, the bands or spots must be stained with a dye. Some of the most commonly used dyes for visualization are Coomassie blue, Fast green, and silver stains.

The separated protein bands or spots may also be visualized by labeling with a fluorophore. In this procedure, the gel is stained with a fluorescent dye, such as ethidium bromide, and illuminated by a light source that is capable of exciting the dye-protein complex in the sample with light at or near the wavelength of the maximum absorbance of the dye-protein complex. The ultraviolet excitation of known dyes typically occurs between about 254-370 nm, while visible excitation occurs at 490-550 nm.

In order to stain, the gel support is first immersed in a solution of the dye and then washed, rinsed, or subjected to some other procedure, to remove the dye from those regions of the support that do not contain protein. The sensitivity of the method of detection depends, among other factors, on the affinity of the dye for the protein and on the difference in visibility between the stained protein regions and the support. The difference in visibility often depends on how well the gel is destained but excessive destaining can result in loss of the protein band signal. Moreover, the post-electrophoresis manipulation of the gels for staining and destaining is inconvenient and costs time and money. There is, therefore, a need for an improved method of staining proteins fractionated by electrophoresis.

The identification, separation, and analysis of particular proteins or subsets of proteins from complex samples are crucial for unravelling how biological processes occur at a molecular level or establishing the degree to which proteins differ among various cell types or between physiological states. A major challenge in modern biology is obtaining an understanding of the expression, function, and regulation of the entire set of proteins encoded by an organism, a technical field commonly known as proteomics. However, as there is no possibility of amplifying proteins, research in this field is generally rather tedious because even a cell extract of a relatively simple prokaryotic organism contains a multitude of proteins encompassing a huge range of concentrations. Such a task is beyond the capabilities of current single analytical methods.

Because of these the methodological constraints, proteome analysis relies not only on methods for identifying and quantifying proteins but—to a considerable extent—also on methods allowing their accurate and reliable separation according to their structural and/or functional properties, the resulting subsets being then better accessible to further analysis. The proteome is of dynamic nature and is subject to alterations in protein synthesis, activation, and/or post-translational modification in response to external stimuli or alterations in the cellular environment. The proteome's inherent complexity thus exceeds that of the genome or the transcriptome the mRNA complement of a cell.

Due to the extraordinary amount of data to be processed in such proteomic studies, the protein/peptide identification process demands tremendous resolving power. Two methods commonly used to resolve such highly complex mixtures are two-dimensional gel electrophoresis (2D-GE; cf., e.g. O'Farrell, P. H. (1975) J. Biol. Chem. 250, 4007-4021) and (two-dimensional) liquid chromatography ((2D)-LC; cf., e.g. Lipton, M. S. et al. (2002) Proc. Natl. Acad. Sci. USA 99, 11049-11054). The peptides and proteins isolated by 2D-GE or 2D-LC are usually identified by mass spectrometry or by determining amino acid composition and/or amino acid sequence. The two-dimensional polyacrylamide gel electrophoresis (2D-GE) is a very sensitive method of separation and will provide resolution of most of the proteins in a sample. Proteins migrate in one- or two-dimensional gels as bands or spots, respectively. The separated proteins are visualized by a variety of methods: by staining with a protein specific dye, by protein mediated silver precipitation, autoradiographic detection of radioactively labeled protein, or by covalent attachment of fluorescent compounds.

However, although useful for many applications, these identification techniques have major drawbacks in proteomic studies where highly complex samples are to be investigated. For example, the detection (sensitivity) limits of these methods as well as shortcomings in labeling technology do not allow for reliable analysis of multiple samples in parallel, e.g. for comparing relative protein levels between different disease groups, different progression stages of a disease, and between disease stages vs. healthy controls or for performing high-throughput screening analyses.

For decades, polyacrylamide gel electrophoresis and related blotting techniques have formed the core technologies for protein analysis. Traditionally, these technologies have been paired with chromogenic dye-based protein detection techniques, such as silver or Coomassie brilliant blue staining. However, with the rapid growth of proteomics, the limitations and experimental disadvantages of absorption-based detection technologies and labor-intensive silver staining techniques have become glaringly apparent. The field of proteomics requires new, highly quantitative electrophoresis and blotting techniques that can interface seamlessly with improved microanalysis methods and that can perform in an increasingly high-throughput environment. These requirements are particularly important for quantitative proteomics and a multiplexed proteomics technology.

Therefore, it is highly desirable to develop methodologies which overcome the above limitations and enable processing of multiple complex samples in parallel.

To compare samples of proteins from different cells or different stages of cell development by conventional methods, each different sample is presently run on separate lanes of a one-dimensional gel or separate two-dimensional gels. Comparison is by visual examination or electronic imaging, for example, by computer-aided image analysis of digitized one or two dimensional gels.

Two-dimensional gel electrophoresis has been a powerful tool for resolving complex mixtures of proteins. The differences between the proteins, however, can be subtle. Imperfections in the gel can hinder accurate observations. In order to minimize the imperfections, the gels provided in commercially available electrophoresis systems are prepared with exacting precision. But even with meticulous controls, no two gels are identical. The gels may differ from each other in pH gradients or uniformity. In addition, the electrophoresis conditions from one run to the next may be different.

Hence, there is a need to eliminate the problems associated with gel distortions and to provide a simple, relatively fast and reliable method of comparing the protein content of different samples in one gel.

Another important facet of protein analysis in general and proteomics in particular relates to the possibility of studying post-translational protein modifications, which can affect activity and binding of a protein and alter its role within the cell (cf., e.g. Pandey, A. and Mann, M. (2000) Nature 405, 837-846). For example, the (reversible) phosphorylation of proteins is crucial for the regulation of many signal transduction cascades such as G-protein-coupled receptor signaling or phospho-tyrosine kinase signaling, whereas an ubiquitination, among others, labels proteins for degradation. Currently, there are several techniques available, for example mass spectrometry, which can in principle distinguish between analogous proteins or peptides based on the presence or absence of a specific modification. However, the available methods are generally hampered by the need to use specific antibodies or other reagents which might in turn interfere with further analyses. Such methods are therefore not particularly suitable for processing multiple samples in parallel and it would thus be desirable to provide methods that allow performance of multiplexed analyses.

WO 96/33406 (Minden, J. and Waggoner, A. S.) describes a method whereby different cell samples are lysed and the total cellular proteins extracted. The different protein samples are then labelled with dyes that are matched for molecular mass and charge to give equivalent migration in 2D electrophoresis. The approach employs cyanine dyes having an N-hydroxysuccinimidyl (NHS) ester reactive group to label amines. The fluorescent pre-labelling of protein samples allows multiple samples to be run on the same gel, thereby enabling quantitative differences between the samples to be identified by overlaying the fluorescent images. However, selection and synthesis of the matched set of dyes is very important since the matched dyes have to be similar in size. Furthermore, proteins that two cell groups have in common form coincident spots in a gel, thereby rendering the results susceptible to undesirable crosstalk between different fluorescent dyes. It is thus highly desirable to develop methodologies which overcome the above limitations.

Considering the limitations of conventional detection methods as outlined above, there is an obvious demand for an alternative method for the qualitative and/or quantitative determination of a proteinaceous molecule in a plurality of samples in order to circumvent the drawbacks of traditional methods.

### OBJECT AND SUMMARY OF THE INVENTION

It is an object of the present invention to provide novel approaches for the qualitative and/or quantitative determination of a proteinaceous molecule in a plurality of samples.

The present invention introduces a method as well as a kit and the use of a set of luminescent dyes for the qualitative and/or quantitative determination of a proteinaceous molecule in a plurality of samples by means of electrophoresis. The present invention thus provides the possibility for a simple, relatively fast and reliable method and kit for comparing the protein content of different samples in parallel.

The invention presented here fulfils the following requirements in an excellent manner, whereas other methods and kits known from the literature lack at least one, mostly more than one of the parameters:
Differences in the amount and/or post-translational modification of a proteinaceous molecule within two separate samples or more can be qualitatively and/or quantitatively determined in one single electrophoresis gel.
A large diversity of commercially available dyes can be used, facilitating easy handling of the dyes and low-cost determination of proteinaceous molecules.
Labeled proteinaceous molecules that two or more samples have in common show a different electrophoretic mobility and fluoresce differently, thereby making it possible to differentiate between those proteinaceous molecules.
Labeled proteinaceous molecules that two or more samples have in common differ with regard to their molecular weight and as such do not form coincident spots in a gel, thereby extenuating the problem of undesirable crosstalk between the fluorescent dyes.

Researchers studying various aspects of cell biology use a variety of tools to detect and monitor differences in cell structure, function and development. An essential part of cell study is the investigation of the differences and similarities in the protein composition between the different cell types, stages of development and condition. Determining differences in the protein content between normal and cancerous cells or wild type and mutant cells, for example, can be a valuable source of information and a valuable diagnostic tool. The method and the kit according to the invention can thus be used in the following fields of application:
a) as a biological tool for studying the protein content of different samples in parallel;
b) as a biological tool for studying post-translational protein modifications in different samples in parallel;
c) as a diagnostic tool for comparing the protein content in different patient samples with standard values and identifying significant deviations which are connected to a particular disease.

These objectives as well as others which will become apparent from the ensuing description are attained by the subject matter of the independent claims. Some of the preferred embodiments of the present invention are defined by the subject matter of the dependent claims.

In one embodiment, the present invention relates to a method for qualitative and/or quantitative determination of a proteinaceous molecule, the method comprising:
(a) providing a plurality of samples, wherein at least one sample contains the proteinaceous molecule;
(b) contacting the proteinaceous molecule with a dye chosen from a set of dyes to label the proteinaceous molecule, wherein each sample is provided with a different dye, and wherein each dye within the set of dyes emits luminescent light at a wavelength that is sufficiently different from the emitted luminescent light of the remaining dyes in the set of dyes to provide a different light signal;
(c) combining the samples;
(d) fractionating the combined samples by means of electrophoresis, wherein the labeled proteinaceous molecule has a relative electrophoretic mobility that differs from the electrophoretic mobility of the proteinaceous molecule labeled with another dye within the set of dyes;
(e) capturing separate images of the labeled proteinaceous molecule at the different wavelengths of emitted luminescence;
(f) aligning the captured images of the labeled proteinaceous molecule by means of image processing; and
(g) obtaining a value indicative for the differences in the amount of the labeled proteinaceous molecule within the plurality of samples.

In another embodiment, the present invention relates to a method for qualitative and/or quantitative determination of a proteinaceous molecule, the method comprising:
(a) providing a plurality of samples, wherein at least one sample contains the proteinaceous molecule;
(b) contacting the proteinaceous molecule with two different dyes chosen from a set of dyes to label the proteinaceous molecule, wherein each sample is provided with a different dye chosen from the set of dyes and is provided with an additional dye from the set of dyes which is provided to all samples, and wherein each dye within the set of dyes emits luminescent light at a wavelength that is sufficiently different from the emitted luminescent light of the remaining dyes in the set of dyes to provide a different light signal;
(c) combining the samples;
(d) fractionating the combined samples by means of electrophoresis, wherein the labeled proteinaceous molecule has a relative electrophoretic mobility that differs from the electrophoretic mobility of the proteinaceous molecule labeled with another dye within the set of dyes;
(e) capturing separate images of the labeled proteinaceous molecule at the different wavelengths of emitted luminescence;
(f) aligning the captured images of the labeled proteinaceous molecule by means of image processing; and
(g) obtaining a value indicative for the differences in the amount of the labeled proteinaceous molecule within the plurality of samples.

In a preferred embodiment, each sample of the plurality of samples contains the proteinaceous molecule.

In another preferred embodiment, the samples are selected from the group consisting of cellular extracts, body fluids and body tissue.

Preferably, the proteinaceous molecule is selected from the group consisting of proteins and peptides.

Also preferably, the dyes are configured so as to covalently bind to at least one binding site of the proteinaceous molecule to label the proteinaceous molecule.

In another embodiment of the inventive method, the labeling reaction comprises contacting the proteinaceous molecule with the dye for a period of time sufficient to form a covalent bond between the dye and the at least one binding site of the proteinaceous molecule.

The at least one binding site can be selected from the group consisting of amino, carboxy and sulfhydryl groups.

In another embodiment, the dyes covalently bind to at least one lysine residue in the proteinaceous molecule.

Preferably, each dye comprises a reactive group which is selected from the group consisting of isothiocyanate, maleimide and N-hydroxysuccinimide.

In another embodiment, each dye within the set of dyes has a molecular weight that differs from the molecular weight of the remaining dyes in the set of dyes.

In a preferred embodiment, at least one dye within the set of dyes has a molecular weight that differs from the molecular weight of the remaining dyes in the set of dyes by at least 100 g/mol, preferably at least 150 g/mol, more preferably at least 200 g/mol and most preferably at least 250 g/mol.

Preferably, each dye has a net charge which will maintain the overall net charge of the proteinaceous molecule upon labeling the proteinaceous molecule.

Also preferably, the labeled proteinaceous molecule has a overall net charge that does not substantially differ from the overall net charge of the unlabeled proteinaceous molecule.

In a further preferred embodiment, each dye within the set of dyes has a hydrophobicity that does not substantially differ from the hydrophobicity of the remaining dyes in the set of dyes.

In another embodiment, the method further comprises, prior to combining the samples, the step of quenching the labeling reaction.

The electrophoresis can be selected from the group consisting of continuous flow electrophoresis, immunoelectrophoresis, moving boundary electrophoresis, paper electrophoresis, polyacrylamide gel electrophoresis and zone electrophoresis.

Preferably, polyacrylamide gel electrophoresis is selected from the group consisting of one-dimensional and two-dimensional polyacrylamide gel electrophoresis.

In another embodiment, capturing separate images of the labeled proteinaceous molecule comprises

(i) capturing a first image of the labeled proteinaceous molecule using a first filter or filters that only allow(s) the passage of light having the wavelength of the luminescent light emitted by a first dye within the set of dyes;
(ii) capturing at least one further image of the labeled proteinaceous molecule using another filter or filters that only allow(s) the passage of light having the wavelength of the luminescent light emitted by another dye within the set of dyes.

In a further embodiment, the method further comprises normalizing the captured images to a common intensity range.

In a preferred embodiment, aligning the captured images of the labeled proteinaceous molecule comprises adjusting the position of the labeled proteinaceous molecule using image processing operations, whereupon the labeled proteinaceous molecule has a relative electrophoretic mobility that is the same as the electrophoretic mobility of the proteinaceous molecule labeled with another dye within the set of dyes.

Preferably, the image processing operations comprise processing the captured images with a computer.

In another embodiment of the inventive method, obtaining a value indicative for the differences in the amount of the labeled proteinaceous molecule within the plurality of samples comprises processing the aligned images of the labeled proteinaceous molecule to determine the difference in luminescent intensity.

Preferably, processing the aligned images of the labeled proteinaceous molecule comprises performing arithmetic operations on values representative of pixel intensities in the aligned images of the labeled proteinaceous molecule.

In another embodiment, the present invention also relates to a kit for use in qualitatively and/or quantitatively determining a proteinaceous molecule in a plurality of samples by means of electrophoresis, wherein at least one sample contains the proteinaceous molecule, the kit comprising: a set of luminescent dyes, wherein each dye within the set of dyes emits luminescent light at a wavelength that is sufficiently different from the emitted luminescent light of the remaining dyes in the set of dyes to provide a different light signal, and wherein each dye within the set of dyes is configured so as to label the proteinaceous molecule, the labeled proteinaceous molecule having a relative electrophoretic mobility that differs from the electrophoretic mobility of the proteinaceous molecule labeled with another dye within the set of dyes.

Preferably, the kit further comprises at least one electrophoresis gel or at least one electrophoresis gel set. Also preferably, the kit further comprises materials for quenching the labeling reaction.

In another embodiment, the present invention also relates to the use of a set of luminescent dyes for the qualitative and/or quantitative determination of a proteinaceous molecule in a plurality of samples by means of electrophoresis, wherein each dye within the set of dyes emits luminescent light at a wavelength that is sufficiently different from the emitted luminescent light of the remaining dyes in the set of dyes to provide a different light signal, is configured so as to label the proteinaceous molecule, the labeled proteinaceous molecule having a relative electrophoretic mobility that differs from the electrophoretic mobility of the proteinaceous molecule labeled with another dye within the set of dyes.

Other embodiments of the present invention will become apparent from the detailed description hereinafter.

### FIGURE LEGENDS

FIG. 1 depicts a schematic illustration of the application of a preferred embodiment of the invention. A general workflow of a proteome analysis in parallel by using 2-D fluorescence gel electrophoresis is shown. Firstly, protein samples are prepared, for example by extraction from frozen and ground plant material. For example, three activated dyes in the form of NHS(N-hydroxysuccinimide) are provided to compose a set of dyes (named “DyeS”, “DyeM” and “DyeL”) and the proteins contained in the samples are labeled with the dyes, wherein an internal standard is composed of equal parts of all samples of one experiment and is labelled with the fluorescence dye DyeS. Each sample is provided with a different dye. The labelled protein samples can then used for the preparation of e.g. a 2D multiplex fluorescence gel. The samples are combined and are subjected to IEF and SDS gel electrophoresis. When digitalizing the 2D multiplex fluorescence gel, the 2D multiplex fluorescence gel provides three pictures after scanning, each picture representing the protein pattern of the used protein sample. Because of the migration differences of identical but differently labelled proteins during SDS gel electrophoresis, these patterns are not exactly congruent to each other. By what is known as intra-gel warping, the position of the protein spots of the DyeM and DyeL dye channel is then corrected with regard to the protein pattern of the DyeS dye gel (reference). On the basis of this correction, spot detection is carried out. This permits the generation of complete expression profiles for the detected spots.

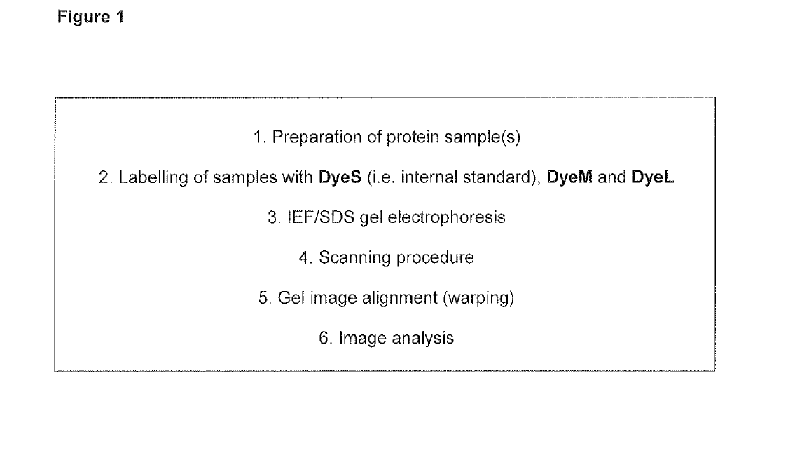

FIG. 2 depicts another schematic illustration of the application of a preferred embodiment of the invention. Protein labeling with three fluorescent dyes of different molecular weight is shown. Dye 1 has a low molecular weight and absorption/emission properties A. Dye 2 has a medium molecular weight and absorption/emission properties B. Dye 3 has a large molecular weight and absorption/emission properties C. Each dye within the set of dyes emits luminescent light at a wavelength that is sufficiently different from the emitted luminescent light of the remaining dyes in the set of dyes to provide a different light signal. Furthermore, each dye within the set of dyes has a molecular weight that differs from the molecular weight of the remaining dyes in the set of dyes.

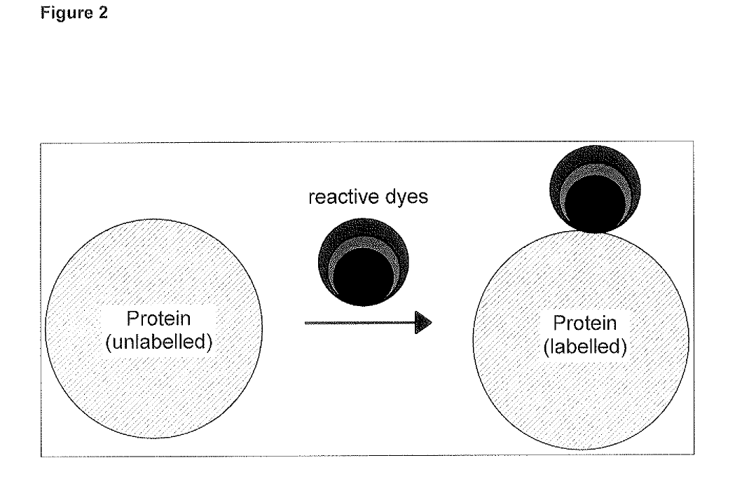

FIG. 3 depicts simulated protein shifts in a two-dimensional electrophoresis gel, wherein three different fluorescent dyes have been used in the labeling reaction (having a molecular weight of 300, 600 and 900 g/mol, respectively). Lane A) without and lane B) with alignment of protein spots in one dimension. Grey spot: unlabelled protein, circles: position of the labelled protein, black spot: aligned positions of the labeled proteins.

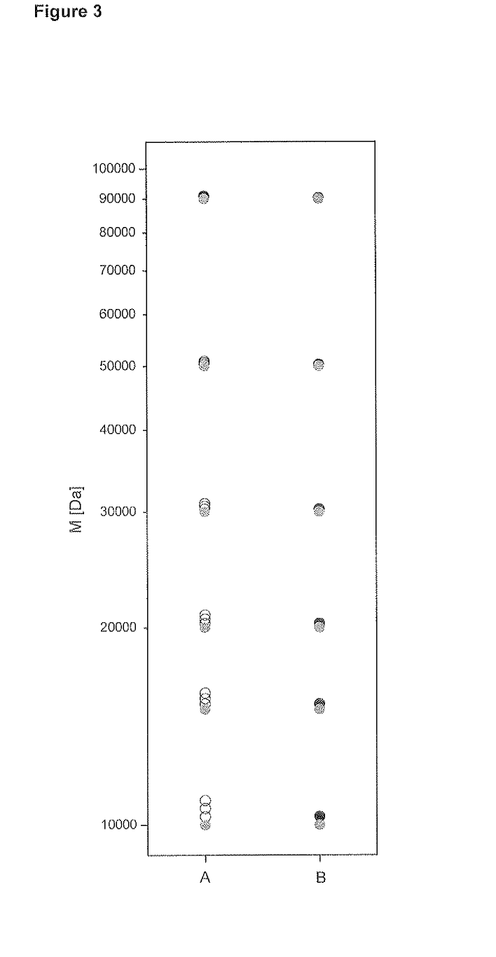

FIG. 4 depicts the process of inter-gel warping of two images from different 2D multiplex fluorescent gels. Figure A) before and B) after warping the images.

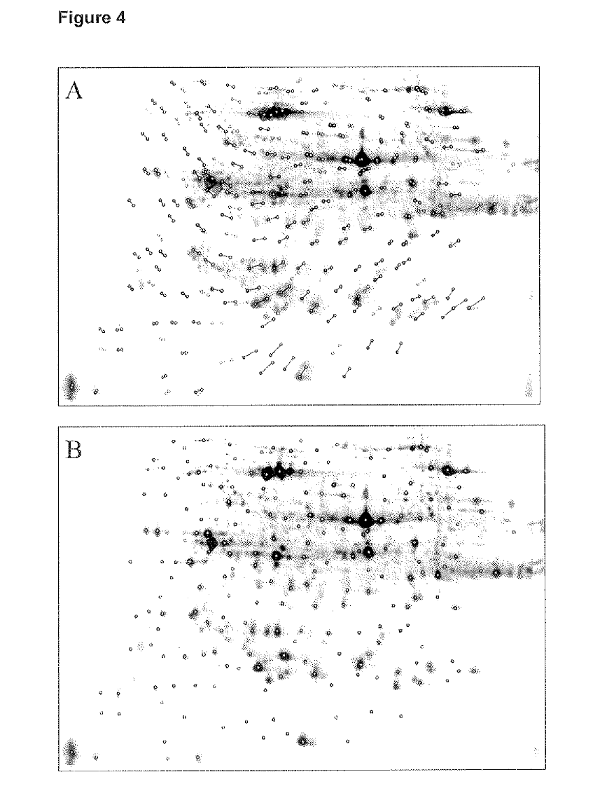

FIG. 5 depicts the process of image alignment (warping) of DyeS fluorescence and silver staining of the same 2D multiplex fluorescence gel. Figure A) before and B) after warping the images.

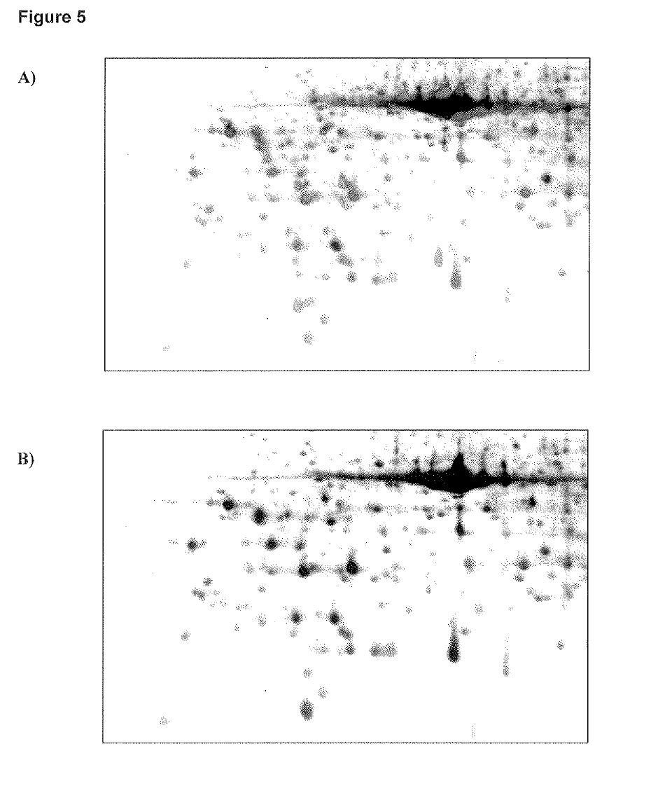

FIG. 6 depicts another schematic illustration of the application of a preferred embodiment of the invention. Each sample out of four samples is provided with two dyes chosen from a set of dyes, wherein one dye is used as internal standard (DyeS). The first two samples are each provided with a different dye chosen from the set of dyes (e.g., DyeM in sample A, DyeL in sample B, etc.) and are additionally provided with another dye chosen from the set of dyes (e.g., DyeS in samples A and B, etc.). Subsequently, equal parts of the first sample duo are combined and are fractionated by means of electrophoresis (e.g., “gel 1”). Similarly, the other two samples are again each provided with a different dye chosen from the same set of dyes (e.g., DyeM in sample C, DyeL in sample D, etc.) and are additionally provided with another dye chosen from the same set of dyes (e.g., DyeS in samples C and D, etc.) which is provided to all samples as an internal standard. Subsequently, equal parts of the further sample duo are combined and are fractionated by means of electrophoresis (e.g., “gel 2”). Furthermore, equal parts of all samples are also combined (e.g., DyeM and DyeS in sample A, DyeL and DyeS in sample B, DyeM and DyeS in sample C, DyeL and DyeS in sample D, etc.) and the combined samples are fractionated by means of electrophoresis (i.e. “gel 3”). An exemplary image analysis and statistical analysis is illustrated.

FIG. 7 depicts the chemical formula of activated DyeS in the form of NHS(N-hydroxysuccinimide).

### DETAILED DESCRIPTION

The present invention is based on the unexpected finding that combining a particular labeling and fractionation strategy with image processing allows the rapid qualitative and/or quantitative determination of a proteinaceous molecule in a plurality of samples. The method is suitable for studying the differences and similarities in the protein composition between different samples in parallel. Since proteinaceous molecules that two samples have in common do not form coincident spots in electrophoresis gels, the method according to the present invention facilitates discrimination between proteinaceous molecules common to two or more samples.

The method and the kit according to the present invention permit the use of a large diversity of dyes. Hence, depending on the application, an individual set of dyes can be easily arranged. This allows the selection of reasonable-priced dyes and/or photostable dyes which are easy to handle and stable during long term storage. Since proteinaceous molecules that two samples have in common do not form coincident spots in electrophoresis gels, undesirable crosstalk between fluorescent dyes is substantially irrelevant when capturing separate images of the labeled proteinaceous molecule at the different wavelengths of emitted luminescence. Crosstalk in fluorescence imaging occurs when the excitation and/or emission spectra of two or more fluorophores (and/or autofluorescence) in a specimen overlap, making it difficult to isolate the activity of one fluorophore alone. Crosstalk can occur during both excitation and emission of different fluorescent proteins and is usually observed by the emission of one fluorophore being detected through the photomultiplier channel or filter combination of another fluorophore. This can be a serious problem in image interpretation, for example in quantitative studies. Crossover in excitation tends to occur increasingly towards shorter wavelengths while crosstalk in emission is skewed towards longer wavelengths. Since proteinaceous molecules that two samples have in common do not form coincident spots in electrophoresis gels, the method according to the present invention substantially eliminates the problem of undesirable crosstalk between fluorescent dyes.

Furthermore, the skilled person has a large variety of possibilities to label the proteinaceous molecule with the dye. Since the method according to the present invention can be conducted independently on the labeling reaction, the method can be used in a large variety of different applications. For example, it can be used as part of a diagnostic method or as a biological tool to study post-translational protein modifications in different samples. Furthermore, the method and the kit according to the present invention facilitate very sensitive labeling reactions, thereby making it possible to detect even marginal differences in the protein composition between different samples in parallel.

Furthermore, it has also been surprisingly found that the method and the kit according to the present invention facilitate easier and more accurate spot picking of individual spots in a two-dimensional polyacrylamide gel electrophoresis in order to investigate a protein spot via mass spectrometry. Since an accurate determination of the coordinates of the respective spot can be easily accomplished, spot picking can be substantially facilitated without loss of yield.

The present invention illustratively described below may suitably be practiced in the absence of any element or elements, limitation or limitations, not specifically disclosed herein.

The present invention will be described with respect to particular embodiments and with reference to certain drawings, although it is not limited thereto but only by the claims. The drawings described are only schematic and are non-limiting. In the drawings, the size of some of the elements may be exaggerated and not drawn to scale for illustrative purposes.

Where the term “comprising” is used in the present description and claims, it does not exclude other elements or steps. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun e.g. “a” or “an”, “the”, this includes a plural of that noun unless otherwise specifically stated.

The term “about” in the context of the present invention denotes an interval of accuracy that the person skilled in the art will understand to still ensure the technical effect of the feature in question. The term typically indicates deviation from the indicated numerical value of ±10%, and preferably ±5%.

Furthermore, the terms first, second, third and the like in the description and in the claims are used to distinguish between similar elements and not necessarily to describe a sequential or chronological order. It is to be understood that the terms so used are interchangeable under appropriate circumstances and that the embodiments of the invention described herein are capable of operation in sequences other than described or illustrated herein.

Further definitions of terms will be given below in the context of which the terms are used.

In one embodiment, the present invention relates to a method for qualitative and/or quantitative determination of a proteinaceous molecule, the method comprising:
(a) providing a plurality of samples, wherein at least one sample contains the proteinaceous molecule;
(b) contacting the proteinaceous molecule with a dye chosen from a set of dyes to label the proteinaceous molecule, wherein each sample is provided with a different dye, and wherein each dye within the set of dyes emits luminescent light at a wavelength that is sufficiently different from the emitted luminescent light of the remaining dyes in the set of dyes to provide a different light signal;
(c) combining the samples;
(d) fractionating the combined samples by means of electrophoresis, wherein the labeled proteinaceous molecule has a relative electrophoretic mobility that differs from the electrophoretic mobility of the proteinaceous molecule labeled with another dye within the set of dyes;
(e) capturing separate images of the labeled proteinaceous molecule at the different wavelengths of emitted luminescence;
(f) aligning the captured images of the labeled proteinaceous molecule by means of image processing; and
(g) obtaining a value indicative for the differences in the amount of the labeled proteinaceous molecule within the plurality of samples.

The term “proteinaceous molecule” as used herein denotes a macromolecular biological substance which comprises a protein or peptide, or a salt or derivative thereof. A proteinaceous molecule may also be a protein or peptide which has been modified by, for example, glycosylation, myristilation, phosphorylation, the addition of lipids, by homologous or heterologous di- or multimerization, or any other (posttranslational) modifications known in the art.

The term “proteins” as used herein refers to any naturally occurring or synthetic (e.g. generated by chemical synthesis or recombinant DNA technology) macromolecules comprising a plurality of natural or modified amino acids connected via peptide bonds.

The length of such molecules may vary from two to several thousand amino acids (the term thus also includes what is generally referred to as oligopeptides). Typically, the term “proteins” relates to molecules having a length of more than 20 amino acids. Thus, proteins to be analyzed in the present invention may have a length from about 30 to about 2500 amino acids, from about 50 to about 1000 amino acids or from about 100 to about 1000 amino acids.

The term “peptide” as used herein refers to any fragments of the above “proteins” that are obtained after cleavage of one or more peptide bonds. A peptide as used in the present invention is not limited in any way with regard to its size or nature. Typically, peptides to be analyzed in the present invention may have a length from about 2 to about 20 amino acids, from about 3 to about 18 amino acids or from about 5 to about 15 amino acids.

Preferably, the proteinaceous molecule is selected from the group consisting of proteins and peptides.

The term “post-translational modification” as used herein denotes any type of chemical modification of a protein and/or peptide according to the invention that takes place after completion of protein translation. Examples of such modifications include inter alia phosphorylation, ubiquitinylation, acetylation, glycosylation, alkylation, isoprenylation, and lipoylation, with phosphorylation being particularly preferred. The term is also to be understood as not to be limited with regard to the numbers and/of types of post-translational modifications being comprised in a protein and/or peptide. Thus, a given protein may comprise in its sequence two or more phosphorylated amino acids or one or more phosphorylated amino acids and one or more ubiquitinylated amino acid residues.

The terms “phospho-proteins” and “phospho-peptides” as used herein denote any proteins and/or peptides comprising in their primary sequence one or more phosphorylated amino acid residues, particularly phosphorylated serine, threonine or tyrosine residues. Within the scope of the present invention, amino acid phosphorylation may occur in vivo by post-translational protein modification or in vitro by employing specific protein kinases.

A “salt” in the context of the invention is generally defined as the product formed from the neutralisation reaction of acids and bases. Salts are ionic compounds composed of cations (positively charged ions) and anions (negative ions) so that the product is electrically neutral (without a net charge). These component ions can be inorganic such as chloride, as well as organic such as acetate and monoatomic ions such as fluoride, as well as polyatomic ions such as sulfate. There are several varieties of salts. Salts that produce hydroxide ions when dissolved in water are basic salts and salts that produce hydronium ions in water acid salts. Neutral salts are those that are neither acid nor basic salts. Zwitterions contain an anionic center and a cationic center in the same molecule but are not considered to be salts. Common salt-forming cations include ammonium, calcium, iron, magnesium, potassium, pyridinium, quaternary ammonium and sodium. Common salt-forming anions (and the name of the parent acids in parentheses) include: acetate (acetic acid), carbonate (carbonic acid), chloride (hydrochloric acid), citrate (citric acid), hydroxide (water), nitrate (nitric acid), oxide (water), phosphate (phosphoric acid), succinate (succinic acid), maleate (maleinic acid), trishydroxymethylaminomethane (tris) and sulfate (sulfuric acid).

The term “derivative” means a chemically modified molecule wherein the chemical modification takes place at one or more functional groups of the compound.

In preferred embodiments, the method further comprises cleaving the proteins into peptides prior to subjecting them to fractionation. Such cleaving of proteins may be achieved either chemically (e.g. via acid or base treatment employing chemicals such as cyanogen bromide, 2-(2′-nitrophenylsulfonyl)-3-methyl-3-bromo-indolenine (BNPS), formic acid, hydroxylamine, iodobenzoic acid, and 2-nitro-5-thiocyanobenzoid acid) or enzymatically via proteases (including inter alia trypsin, pepsin, thrombin, papain, and proteinase K) well known in the art.

The term “sample” as used herein is not intended to necessarily include or exclude any processing steps prior to the performing of the methods of the invention. The samples can be unprocessed (“crude”) samples, extracted protein fractions, purified protein fractions and the like. For example, the samples employed may be pre-processed by immunodepletion of one or more subsets of abundant proteins. Suitable samples include samples of prokaryotic (e.g. bacterial, viral samples) or eukaryotic origin (e.g. fungal, yeast, plant, invertebrate, mammalian and particularly human samples). Preferably, the samples are selected from the group consisting of cellular extracts, body fluids and body tissue.

The term “complex sample” as used herein denotes that a sample analyzed using a method of the present invention typically includes a multitude of different proteins and/or peptides (or different variants of such proteins and/or peptides) present in different concentrations. For example, complex samples within the present invention may include at least about 500, at least about 1000, at least about 5000 or at least about 10000 proteins and/or peptides. Typical complex samples used in the invention include inter alia cell extracts or lysates of prokaryotic or eukaryotic origin as well as human or non-human body fluids such as whole blood, serum, plasma samples or the like.

The term “plurality of samples” as used herein denotes at least two samples. In a preferred embodiment, each sample of the plurality of samples contains the proteinaceous molecule. However, it is also possible to perform the method according to the present invention if the proteinaceous molecule is not present in each of the samples (e.g. when monitoring inhibition or stimulation of protein expression). The plurality of samples can be any two or more sets of cells, the protein content of which one wishes to compare or contrast. For example, the first group of cells can be the wild-type or normal cells, and the second group of cells can be mutant cells from the same species. Alternatively, the first group of cells can be normal cells and the second group can be cancerous cells from the same individual. Cells from the same individual at different stages of development or different phases of the cell cycle can be used also. The differences in protein composition between cells of the same type from different species can also be the subject of study using the process of the present invention. In addition, the process of the present invention can be used to monitor how cells respond to a variety of stimuli or drugs. Hence, it is possible to monitor the stimulation of the expression of a protein after providing a group of cells with an stimulant, but it is also possible to monitor the inhibition of the expression of a protein after providing a group of cells with an inhibitor.

The term “cellular extracts” as used herein denotes extracts from cell populations. In the context of the present invention, proteinaceous molecules can be extracted from cell populations using an extraction medium that comprises a salt and a detergent. The cell populations may comprise any cell or virus that comprises proteinaceous molecules (e.g. proteins or peptides). The cell may be any of a variety of different types of cells including, for example, eukaryotic cells such as fungal, protist, plant or animal cells. Preferably the cell is a mammalian cell, such as a rodent, mouse, rat, hamster, primate or human cell. The cells may be living, dead, or damaged, that is, having disruptions in the cell wall or cell membrane. The cell may be obtained from any source, as will be understood by those of skill in the art, including from a cell culture, from a sample collected from a subject (e.g. from an animal, including a human) or the environment, from a tissue sample or body fluid (e.g. whole blood, plasma, serum, urine, or cerebral spinal fluid), and other such sources.

The cell population may be directly contacted with the extraction medium, or alternately, the cell population may be first concentrated by methods such as centrifugation, binding to a surface through immunoadsorption or other interaction, or filtration, prior to contact with the extraction medium. Optionally, the number of cells in the population may be increased by growing the cells on culture plates or in a suitable liquid medium prior to concentration or direct extraction. Methods and media for growing cells are well known to those of skill in the art.

In a preferred embodiment of the present invention, the cell population is prepared by harvesting the cells, and optionally washing the cells to remove contaminants prior to contacting the cells with the extraction medium. For example, for cells in a cell suspension, the cells may be harvested from the growth medium by trypsinization or by centrifugation at a force of from about 1 to about 100,000×g, more preferably at a force of from about 100 to about 10,000×g, and preferably about 300×g for about 0.01 to about 1500 minutes, and preferably for about 5 minutes. The growth medium may be removed by any suitable method including, for example, aspiration. In one embodiment, the cell pellet may be washed using a suitable wash solution (e.g. PBS), repelleted as described above, and the wash solution removed by aspiration. The resulting cell population may then be contacted with the extraction medium, as described herein. A similar method may be used to remove the growth media from cells attached to a substrate (e.g. a tube or plate). In this instance, the cells can be contacted directly with extraction media after removal of growth media without the need for harvesting cells via trypsinization and centrifugation.

Once a suitable cell population has been obtained, proteinaceous molecules may be extracted from the cells using an extraction medium. The extraction medium causes the release of proteinaceous molecules from cells present in the sample. In one preferred embodiment, the extraction medium comprises a detergent, a salt, and optionally other components that aid in the extraction, inter alia proteinase inhibitors. Without wishing to be bound by any particular theory, it is believed that the extraction medium ruptures the cells through the action of the detergent and the salt. The detergent aids in the extraction by perforating the cell membrane, while the salt renders the extraction medium hypertonic. Under these hypertonic conditions, proteinaceous molecules are released from the cytosol of the ruptured cells through osmotic pressure exerted on the cell wall and/or cell membrane as the cell collapses in on itself. Suitable methods and buffers for extracting proteinaceous molecules from cells are well known to those of skill in the art (Guide to Protein Purification (M. P. Deutscher, ed), Academic Press, 1990; Protein Methods (D. M. Bollag, M. D. Rozycki, S. J. Edelstein, eds), 2nd ed., Wiley-Liss, New York, 1996; Protein Expression: A Practical Approach (S. J. Higgins and B. D. James, eds), Oxford University Press, Oxford, UK, 1999; Protein Purification, Amersham Pharmacia Biotech handbook, 1999, The Recombinant Protein Handbook; Protein amplification and simple purification, Amersham Pharmacia Biotech handbook, 2000).

The term “dye” as used herein denotes a fluorescent dye. Hereinafter, the terms “fluorescent dyes”, “fluorescent probes” and “fluorescent labels” are used as synonyms. Fluorescence is a luminescence that is mostly found as an optical phenomenon in cold bodies, in which the molecular absorption of a photon triggers the emission of another photon with a longer wavelength. The energy difference between the absorbed and emitted photons ends up as molecular vibrations or heat. Usually the absorbed photon is in the ultraviolet range and the emitted light is in the visible range, but this depends on the absorbance curve and Stokes shift of the particular fluorophore. “Stokes shift” is the difference (in wavelength or frequency units) between positions of the band maxima of the absorption and emission spectra (fluorescence and Raman being two examples) of the same electronic transition.

A “fluorescent dye” comprises a fluorophore which, in analogy to a chromophore, is a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. Fluorescein isothiocyanate, a reactive derivative of fluorescein, has been one of the most common fluorophores chemically attached to other, non-fluorescent molecules to create new and fluorescent molecules for a variety of applications. Other historically common fluorophores are derivatives of rhodamine, coumarin and cyanine Representative fluorophores include derivatives of fluorescein, derivatives of bodipy, 5-(2′-aminoethyl)-aminonaphthalene-1-sulfonic acid (EDANS), derivatives of rhodamine, cyanine dyes, e.g. Cy2, Cy3, Cy 3.5, Cy5, Cy5.5, Cy7, texas red and its derivatives, and derivatives of perylene dyes, terrylene dyes, quaterrylene dyes, naphthalimide dyes, xanthene dyes, oxazin dyes, anthracene dyes, naphthacene dyes, anthraquinone dyes and thiazine dyes.

The term “set of dyes” as used herein denotes a set of fluorescent dyes, the set comprising at least two fluorescent dyes, which are selected with regard to their absorption and emission bands in that each dye within the set of dyes emits luminescent light at a wavelength that is sufficiently different from the emitted luminescent light of the remaining dyes in the set of dyes to provide a different light signal. Since a multicolor labeling experiment entails the deliberate introduction of two or more fluorophores to simultaneously monitor different biochemical functions, an ideal combination of dyes for multicolor labeling would exhibit strong absorption at a coincident excitation wavelength and well separated emission spectra. Fluorophores currently used as fluorescent probes offer sufficient permutations of wavelength range, Stokes shift and spectral bandwidth to meet requirements imposed by instrumentation (e.g. 488 nm excitation), while allowing flexibility in the design of multicolor labeling experiments. Hence, the absorption and fluorescence emission spectral profiles of a fluorophore are two criteria that should be scrutinized when selecting probes for the inventive method.

Suitable fluorescent dyes include, inter alia, ATTO dyes (e.g., ATTO 495 (MW (g/mol): 549); ATTO 550 (MW (g/mol): 791); ATTO 647N (MW (g/mol): 843) sold through Sigma-Aldrich, Inc. These and other suitable fluorescent dyes are well known to those skilled in the art. Many such dyes are described in the Handbook: (The Handbook—A Guide to Fluorescent Probes and Labeling Technologies Web Edition of The Handbook, Tenth Edition 2006 Invitrogen Corporation).

Usually fluorescence dyes with higher brightness (brightness of a fluorochrome is proportional to the product of its extinction coefficient and its quantum efficiency), longer excitation and emission wavelengths, and greater photostability are preferred, but the choice of the dye depends on the analytical instrument used for the assay and particular analytical situation. Fluorescent dyes that may be useful in the present invention include fluorescein and fluorescein derivatives, rhodamine and rhodamine derivatives, derivatives of coumarin, carbopyronin and oxazine, and many other fluorescent dyes that are produced and sold by different companies.

In a preferred embodiment of the invention, the set of dyes comprises three different fluorescent dyes. In such an embodiment, the absorption maximum of the first fluorescent dye is between 390 nm and 520 nm, preferably between 425 nm and 510 nm, more preferably between 465 nm and 505 nm, and most preferably between 488 nm and 500 nm; the absorption maximum of the second fluorescent dye is between 520 nm and 620 nm, preferably between 530 nm and 610 nm, more preferably between 535 nm and 590 nm, and most preferably between 540 nm and 565 nm and the absorption maximum of the third fluorescent dye is between 620 nm and 740 nm, preferably between 630 nm and 725 nm, more preferably between 635 nm and 700 nm, and most preferably between 637 nm and 680 nm.

In another preferred embodiment, the fluorescence maximum of the first fluorescent dye is between 479 nm and 540 nm, preferably between 484 nm and 535 nm, more preferably between 508 nm and 532 nm, and most preferably between 523 nm and 530 nm; the fluorescence maximum of the second fluorescent dye is between 540 nm and 650 nm, preferably between 550 nm and 630 nm, more preferably between 560 nm and 620 nm, and most preferably between 565 nm and 600 nm, and the fluorescence maximum of the third fluorescent dye is between 650 nm and 764 nm, preferably between 655 nm and 752 nm, more preferably between 660 nm and 719 nm, and most preferably between 665 nm and 700 nm.

In another preferred embodiment of the invention, the molecular weight of a first fluorophore comprised by the set of dyes (fluorophore without reactive group) is less than 500 g/mol, preferably less than 450 g/mol, more preferably less than 400 g/mol, and most preferably less than 350 g/mol. Preferably, the molecular weight of a second and/or any further fluorophore comprised by the set of dyes is above 600 g/mol, more preferably above 650 g/mol, and most preferably above 700 g/mol. In a preferred embodiment, a first fluorophore comprised by the set of dyes (fluorophore without reactive group) has a molecular weight that differs from the molecular weight of the further fluorophores comprised by the set of dyes by at least 100 g/mol, preferably at least 150 g/mol, more preferably at least 200 g/mol and most preferably at least 250 g/mol.

In another preferred embodiment, each dye within the set of dyes has a molecular weight that differs from the molecular weight of the remaining dyes in the set of dyes. In a preferred embodiment, at least one dye within the set of dyes has a molecular weight that differs from the molecular weight of the remaining dyes in the set of dyes by at least 100 g/mol, preferably at least 150 g/mol, more preferably at least 200 g/mol and most preferably at least 250 g/mol. Differences between the fluorescent dyes with regard to their molecular weight cause the labeled proteinaceous molecule to have a relative electrophoretic mobility that differs from the electrophoretic mobility of the proteinaceous molecule labeled with another dye within the set of dyes. Hence, labeled proteinaceous molecules that two samples have in common do not form coincident spots in electrophoresis gels. Labeled proteinaceous molecules that two samples have in common are each labeled with a different dye and as such also differ with regard to their molecular weight.

In another preferred embodiment of the invention, the set of dyes comprises two, three or four different fluorescent dyes (depending on the number of samples to be investigated), the fluorescent dyes being selected with regard to their absorption and emission bands in that each dye within the set of dyes emits luminescent light at a wavelength that is sufficiently different from the emitted luminescent light of the remaining dyes in the set of dyes to provide a different light signal. Although the preferred absorption maximum of each of the fluorescent dyes is between 390 nm and 740 nm and the preferred fluorescence maximum of each of the fluorescent dyes is between 479 nm and 764 nm, fluorescent dyes included in the set of dyes may also have longer or shorter wavelength absorption or fluorescence maxima. The choice of the dyes generally depends on the analytical instrument used for the assay and particular analytical situation. However, it is understood that the absorption maximum does not coincide with the molecular weight.

In a further preferred embodiment, the set of dyes comprises two, three or four different fluorescent dyes which are provided to the respective samples in equal amounts or concentrations.

Preferably, each dye has a net charge which will maintain the overall net charge of the proteinaceous molecule upon labeling the proteinaceous molecule. Also preferably, the labeled proteinaceous molecule has an overall net charge that does not differ substantially from the overall net charge of the unlabeled proteinaceous molecule. When e.g. lysine is the attachment site on the proteinaceous molecule, the covalent linkage destroys the positive charge of the primary amine of the lysine. Because e.g. isoelectric focusing depends on charge, it may thus be important to compensate for the charge loss.

In a further preferred embodiment, each dye within the set of dyes has a hydrophobicity that does not substantially differ from the hydrophobicity of the remaining dyes in the set of dyes. The term “hydrophobicity” as used herein refers to the physical property of a molecule that is repelled from a mass of water. Hydrophobic molecules tend to be non-polar and thus prefer other neutral molecules and nonpolar solvents. Examples of hydrophobic molecules include the alkanes, oils, fats, and greasy substances in general. Preferably, all dyes within the set of dyes are moderately hydrophilic. A hydrophilic molecule or portion of a molecule is one that is typically charge-polarized and capable of hydrogen bonding, enabling it to dissolve more readily in water than in oil or other hydrophobic solvents. Hydrophilic and hydrophobic molecules are also known as polar molecules and nonpolar molecules, respectively.

Preferably, the fluorescent dyes are designed to be more stable under prolonged irradiation. Apart from absorption and fluorescence there are many other dye properties that may be relevant with respect to their suitability as labels. For example, the dye should remain intact during irradiation. Many common labels, e.g. fluorescein (FITC), show very low photostability. As a result, sensitivity and imaging quality are limited if high-intensity laser excitation is used.

The molecules of most common dyes, e.g. cyanines, have a more or less flexible structure. Hence, their solutions contain a mixture of several isomers with varying properties. Since the equilibrium between the isomers depends on temperature and other environmental factors, absorption and fluorescence of such dyes often are ill-defined. In a preferred embodiment of the invention, the fluorescent dyes have a molecular structure that ensures high rigidity of the chromophore. Preferably, the fluorescent dyes as used herein do not form equilibria with various isomers, their optical properties are practically independent of solvent and temperature. Preferably, the fluorescent dyes according to the present invention are not cyanine dyes or derivatives thereof, such as Cy2, Cy3, Cy3.5, Cy5, Cy5.5 or Cy7.

Characteristic features of particularly preferred fluorescent dyes are strong absorption, high fluorescence quantum yield, excellent photostability, high ozone resistance and good water solubility.

The method according to the present invention includes labeling of proteinaceous molecules prior to subjecting them to fractionation. The term “labeling” as used herein denotes the attachment or incorporation of one or more detectable dyes (or “labels” or “probes”) into a proteinaceous molecule used in the invention. This is done by contacting the proteinaceous molecule with a dye chosen from a set of dyes, each sample being provided with a different dye. Contacting the proteinaceous molecule with a dye may thus be conducted by adding a different dye to each sample. To label the proteinaceous molecule, the reactive form of the dye and the proteinaceous molecule are incubated for a period of time sufficient to allow for the formation of a tight bond between the reactive form of the dye and the proteinaceous molecule. The period of time is generally from 15 to 60 minutes, depending on the temperature. The temperature range is generally from about 0° C. to 25° C. Hence, proteinaceous molecules that two samples have in common are labeled with a different dye, respectively, and as such are provided a different light signal when excited. Preferably, the labeling reaction comprises contacting the proteinaceous molecule with the dye for a period of time sufficient to form a covalent bond between the dye and the at least one binding site of the proteinaceous molecule.

The term “binding site” as used herein denotes a region on the proteinaceous molecule to which specific other molecules and ions—in this context the fluorescent dyes—form a chemical bond. Preferably, the chemical bond is a covalent bond between a binding site of the proteinaceous molecule and a reactive group of the fluorescent dye.

The ratio of the fluorescent intensity between identical proteinaceous molecules from either sample will be constant for the vast majority of molecules. Thus, a proteinaceous molecule that is unique or of different relative concentration to one sample will have a different ratio of fluorescence intensity from the majority of protein spots, and will produce a color specific for one or other of the samples, depending on the fluorescent dye used. For example, the proteinaceous molecules that are in the first sample may be labeled red, while the proteinaceous molecules of the second sample are labeled blue.

Preferably, the “labeled proteinaceous molecule” has a high fluorescence yield, yet still retains the critical parameters of the unlabeled proteinaceous molecule, such as solubility, selective binding to a receptor or nucleic acid, activation or inhibition of a particular enzyme or the ability to incorporate into a biological membrane. Frequently, however, proteinaceous molecules with the highest degree of labeling precipitate or bind non-specifically. It may therefore be necessary to have a less-than-maximal fluorescence yield to preserve function or binding specificity. Conjugating dyes to proteinaceous molecules is commonly known in the art (The Handbook—A Guide to Fluorescent Probes and Labeling Technologies Web Edition of The Handbook, Tenth Edition 2006 Invitrogen Corporation).

Following conjugation, it may be important to remove as much unconjugated labeling reagent as possible. The presence of free dye, particularly if it remains chemically reactive, can greatly complicate subsequent experiments with the proteinaceous molecule. In a preferred embodiment, the method thus further comprises, prior to combining the samples, the step of quenching the labeling reaction, usually by gel filtration, dialysis, bioconjugate precipitation and resolubilization, HPLC or a combination of these techniques. Alternatively or in combination, any suitable known quenching material may be used. Preferably, the labeling reaction is quenched by the addition of lysine. In situations where the labeling reaction is allowed to go to completion, for example when all the reactive dye has been used, quenching or removal of excess dye may not be required (e.g. saturation labeling).

In another preferred embodiment, the labeled proteinaceous molecule has a molecular weight that differs from the molecular weight of the proteinaceous molecule labeled another dye within the set of dyes. This causes the labeled proteinaceous molecule to have a relative electrophoretic mobility that differs from the electrophoretic mobility of the proteinaceous molecule labeled with another dye within the set of dyes. Hence, proteinaceous molecules that two samples have in common do not form coincident spots in electrophoresis gels.

In one embodiment of the present invention, the fluorescent dyes are covalently coupled to the proteinaceous molecule, preferably via lysine residues, but coupling may also be to sulfhydryl or carboxylic acid groups in the proteinaceous molecule. When a sulfhydryl group is the binding site on the proteinaceous molecule, the corresponding reactive group of the dye preferably is an iodoalkyl group. When a carboxylic acid group is the binding site on the proteinaceous molecule, carbodiimide may be used to convert the carboxylic acid into a reactive intermediate which is then susceptible to attack by amines.

In one embodiment of the present invention, amine-reactive dyes may be used to label the proteinaceous molecules. In contrast to thiol-reactive reagents, which frequently serve as probes of protein structure and function, amine-reactive dyes are most often used to prepare bioconjugates for immunochemistry, fluorescence in situ hybridization (FISH), cell tracing, receptor labeling and fluorescent analog cytochemistry. In these applications, the stability of the chemical bond between the dye and biomolecule is particularly important because the conjugate is typically stored and used repeatedly over a relatively long period of time. Moreover, these conjugates are often subjected to rigorous incubation, hybridization and washing steps that demand a strong dye-biomolecule linkage. Amine-reactive probes are mostly acylating reagents that form carboxamides, sulfonamides or thioureas upon reaction with amines. The kinetics of the reaction depends on the reactivity and concentration of both the acylating reagent and the amine. Of course, buffers that contain free amines such as Tris and glycine must be avoided when using any amine-reactive probe. Ammonium sulfate that has been used for protein precipitation must also be removed before performing dye conjugations. In addition, high concentrations of nucleophilic thiols should be avoided because they may react with the reagent to form an unstable intermediate that could consume the dye.

The most significant factors affecting an amine's reactivity are its class and its basicity. Virtually all proteins have lysine residues, and most have a free amine at the N-terminus. Aliphatic amines such as lysine's ε-amino group are moderately basic and reactive with most acylating reagents. However, the concentration of the free base form of aliphatic amines below pH 8 is very low. Thus, the kinetics of acylation reactions of amines by isothiocyanates, succinimidyl esters and other reagents are strongly pH dependent. A pH of 8.5 to 9.5 is preferred for modifying lysine residues. In contrast, the α-amino group at a protein's N-terminus usually has a pKa of ˜7, so it can sometimes be selectively modified by reaction at near neutral pH. Furthermore, although amine acylation is preferably carried out above pH 8.5, the acylation reagents tend to degrade in the presence of water, with the rate increasing as the pH increases.

In another embodiment of the present invention, thiol-reactive dyes may be used to prepare labeled proteinaceous molecules. Because the thiol functional group is not commonly present in most proteins and can be labeled with high selectivity, thiol-reactive reagents often provide a means of modifying a protein at a defined site. Thiol-reactive dyes can be reacted with thiol-containing proteins to facilitate their electrophoretic detection. In proteins, thiol groups (also called mercaptans or sulfhydryls) are present in cysteine residues. Thiols can also be generated by selectively reducing cystine disulfides with reagents such as dithiothreitol (DTT) or 2-mercaptoethanol (β-mercaptoethanol), each of which must then be removed by dialysis or gel filtration before reaction with the thiol-reactive probe. The common thiol-reactive functional groups are primarily alkylating reagents, including iodoacetamides, maleimides, benzylic halides and bromomethylketones.

Preferably, each dye comprises a reactive group which is selected from the group consisting of isothiocyanate, maleimide and N-hydroxysuccinimide. It is particularly preferred that each dye within the set of dyes comprises the same reactive group.

Succinimidyl esters are excellent reagents for amine modification because the amide bonds they form are as stable as peptide bonds. These reagents are generally stable during storage if well desiccated, and show good reactivity with aliphatic amines and very low reactivity with aromatic amines, alcohols, phenols (including tyrosine) and histidine. Succinimidyl esters will also react with thiols in organic solvents to form thioesters. If formed in a protein, a thioester may transfer the acyl moiety to a nearby amine. Some succinimidyl esters may not be compatible with a specific application because they can be quite insoluble in aqueous solution. To overcome this limitation, carboxylic acid derivatives of fluorophores may used, which can be converted into sulfosuccinimidyl esters or STP esters. These sulfonated reagents have higher water solubility than simple succinimidyl esters and sometimes eliminate the need for organic solvents in the conjugation reaction. Alternatively, isothiocyanates may be used as amine-reactive dyes, forming thioureas upon reaction with amines. Despite the growing number of choices in amine-reactive fluorophores, fluorescein isothiocyanate (FITC) and tetramethylrhodamine isothiocyanate (TRITC) are still widely used reactive fluorescent dyes for preparing fluorescent bioconjugates.

Iodoacetamides readily react with all thiols, including those found in peptides, proteins and thiolated polynucleotides, to form thioethers; they are somewhat more reactive than bromoacetamides. Maleimides are excellent reagents for thiol-selective modification, quantitation and analysis. In this reaction, the thiol is added across the double bond of the maleimide to yield a thioether. Applications of these fluorescent and chromophoric analogs of N-ethylmaleimide (NEM) strongly overlap those of iodoacetamides, although maleimides apparently do not react with methionine, histidine or tyrosine.

Although alcohols (including phenols such as tyrosine and the hydroxyl groups in serine, threonine, sterols and carbohydrates) are abundant in biomolecules, their chemical reactivity in aqueous solution is extremely low. Nevertheless, nonacylated N-terminal serine and threonine residues in peptides and proteins can be oxidized with periodate to yield aldehydes that can be subsequently modified with a variety of hydrazine, hydroxylamine or amine derivatives. Tyrosine residues in some proteins can be selectively modified by initial nitration of the ortho position of its phenol using tetranitromethane, and then reduction of the o-nitrotyrosine with sodium dithionite to form an o-aminotyrosine.

Preferably, the dyes are configured so as to covalently bind to at least one binding site of the proteinaceous molecule to label the proteinaceous molecule. The at least one binding site can inter alia be selected from the group consisting of amino, carboxy and sulfhydryl group. It is further preferred that a plurality of binding sites be labeled. The optimum percentage of binding sites labeled will depend on the dyes and target functional groups chosen. When dyes are used to label lysines, preferably no more than 2% of the attachment sites and more preferably, slightly less than 1%, are labeled. Thus, where a typical protein is composed of about 7% lysines, there will be less than one modified amino acid per one thousand. A typical protein is composed of about 450 amino acids. Almost all large-scale projects in mass spectrometry-based proteomics use trypsin to convert protein mixtures into more readily analyzable peptide populations. Since trypsin cleaves e.g. lysine residues, this “minimal dye labeling” approach will ensure that lysine residues are still available in the labeled protein for subsequent mass spectrometry-based proteomics. An alternative strategy, so called “saturation labeling”, is to label all the functional groups of a particular type which is less prevalent in the protein, for example sulfhydryl groups in cysteines. Both strategies may be likewise applied when performing the method according to the present invention.

Furthermore, when carrying out the inventive method, it is possible to target specific groups of proteins, such as proteins bearing post-translational modifications, in order to compare differences in the postranslational modifications and other differences occurring in proteins between two or more samples.

An example of such proteins is glycoproteins. In recent years, the functional significance of carbohydrate on proteins has been increasingly realized. Carbohydrates are now known to be implicated in many cellular and disease processes. It is possible to label the terminal carbohydrate groups of glycoproteins by first oxidizing the terminal sugars to aldehydes, followed by reaction with an hapten-linker conjugate having a hydrazide reactive group, as described by Wilchek, M and Bayer, E. A., “Methods in Enzymology” 138, 429-442 (1987). Fluorescent dyes that can be used for glycoprotein labeling include those dyes having hydrazine derivatives such as hydrazides, semicarbazides and carbohydrazides or amine derivatives as reactive groups.

According to another aspect of the invention, a method for specifically labeling a phosphorylated proteinaceous molecule is provided. The method involves: (1) subjecting the proteinaceous molecule to conditions so as to effect a beta-elimination reaction in the intact proteinaceous molecule to convert the phosphorylated amino acid to a dehydroamino acid including a double bond containing carbon atoms that are susceptible to attack by an addition reaction; and (2) labeling the proteinaceous molecule of step (1) with a dye under conditions so as to add the dye to the carbon atom of the double bond of the dehydroamino acid to form an amino acid addition product in the intact proteinaceous molecule. By subjecting the labeled proteinaceous molecules to fractionation as described hereinafter the amino acid addition product in the intact proteinaceous molecule can be detected, the presence of the amino acid addition product being indicative of the presence of the phosphorylated amino acid in the intact proteinaceous molecule. Although this particular labeling reaction is not limited in scope to a particular mechanism, it is believed that such addition reactions disclosed herein primarily proceed via nucleophilic addition.

Thus, in a particularly preferred embodiment, the dehydroamino acid double bond is subjected to a nucleophilic addition reaction to form a nucleophilic addition product. The “reactant” in such a nucleophilic addition reaction is referred to herein as a nucleophile and the nucleophilic addition product is referred to herein as a nucleophilic amino acid addition product. Preferably, the nucleophile is a nucleophile which contains a single functional group for covalent attachment to an amino acid residue in an intact proteinaceous molecule and further includes a fluorescent dye for detecting the presence of the phosphorylated amino acid in the intact proteinaceous molecule.

Alternatively, the phosphate groups on proteins may be specifically labeled as, for example, using the procedure described by Giese and Wang, U.S. Pat. No. 5,512,486, incorporated herein by reference, using fluorescent or hapten imidazole derivatives. The labeling reagents bearing the imidazole groups are added to the protein samples in the presence of a suitable carbodiimide to effect labeling.

Since phosphorylated tyrosine residues are not accessible to chemical cleavage via beta-elimination, it is preferred to remove the phosphate group enzymatically. Enzymatic removal may be achieved via phosphatases, particularly via alkaline phosphatases (for the non-specific removal of phosphate-groups) or via protein tyrosine phosphatases (for specifically dephosphorylating phospho-tyrosine residues). Such phosphatases are commercially available.

In a preferred embodiment of the invention, it is recommended that cysteine, cystine and/or lysine residues be modified prior to conducting the beta-elimination to avoid their participation in side reactions which could interfere with the determination of phosphoserine and phosphothreonine residues in the intact protein. Methods for modifying cysteine, cystine and lysine residues are commonly known in the art.

The term “fractionation”, as used herein, generally refers to any type of separation process in which the labeled proteinaceous molecules present in the combined sample in a certain quantity are divided up (i.e. sorted) into a large number of smaller quantities (i.e. fractions) according to any differences in their physico-chemical properties such as the molecular mass, their size, and their overall net charge. A common trait in fractionations is the need to find an optimum between the amount of fractions collected and the desired purity in each fraction. Fractionation makes it possible to isolate more than two components in a mixture in a single run. This sets it apart from other separation techniques. There are several methods for fractionating proteinaceous molecules which are well established in the art. These include classical SDS polyacrylamide gel electrophoresis (SDS-PAGE), two-dimensional gel electrophoresis, size-exclusion chromatography, (two-dimensional) liquid chromatography, and isoelectric focusing. Methods for analysis, particularly visualization, of the proteinaceous molecules after fractionation has taken place are well established in the art.

After labeling, the samples are combined and fractionated by any suitable known fractionation technique, such as electrophoresis, the electrophoresis preferably being selected from the group consisting of continuous flow electrophoresis, immunoelectrophoresis, moving boundary electrophoresis, paper electrophoresis, polyacrylamide gel electrophoresis and zone electrophoresis. Preferably, polyacrylamide gel electrophoresis is selected from the group consisting of one-dimensional and two-dimensional polyacrylamide gel electrophoresis, with the latter one being particularly preferred.

“Isoelectric focusing” is a technique for separating different molecules by their electric charge differences. It is a type of zone electrophoresis, usually performed in a gel (e.g. an agarose gel or, preferably, polyacrylamide gel) that takes advantage of the fact that a molecule's charge changes with the pH of its surroundings. The molecules to be focused are distributed over a medium that has a pH gradient. An electric current is passed through the medium, creating a “positive” anode and “negative” cathode end. Negatively charged molecules migrate through the pH gradient in the medium toward the “positive” end while positively charged molecules move toward the “negative” end. As a particle moves towards the pole opposite of its charge it moves through the changing pH gradient until it reaches a point in which the pH of that molecule's isoelectric point (pI) is reached. At this point the molecule no longer has a net electric charge (due to the protonation or deprotonation of the associated functional groups) and as such will not proceed any further within the gel. Isoelectric focusing can resolve proteins that differ in pI value by as little as 0.01. It is usually the first step in two-dimensional gel electrophoresis, in which labeled proteinaceous molecules are first separated by their pI and then further separated by molecular weight through standard SDS-PAGE.

Depending on any differences in the dyes' physico-chemical properties such as the molecular mass and/or their size, the labeled proteinaceous molecule has a relative electrophoretic mobility that differs from the electrophoretic mobility of the proteinaceous molecule labeled with another dye within the set of dyes. Hence, proteinaceous molecules that two samples have in common differ with regard to their molecular weight as well as their electrophoretic mobility and as such do not form coincident spots in electrophoresis gels.

In a preferred embodiment, when combing the samples, at least one sample comprising the labeled proteinaceous molecule is provided in higher amounts or a higher concentration than the other samples. For example, the labeled proteinaceous molecule may be provided in an amount or a concentration that is two to five, preferably four times higher than the amount or the concentration of the labeled proteinaceous molecule labeled with another dye within the set of dyes. This can have the technical effect of increasing the spot intensity of the proteinaceous molecule labeled with the dye provided in higher amounts or a higher concentration (e.g., DyeS), which may have an advantageous impact on image analysis.

After fractionation, separate images of the labeled proteinaceous molecule at the different wavelengths of emitted luminescence are captured. The gel can be analyzed by a fluorescence scanner, by a fluorescent microscope or by any known means for detecting fluorescence. Gel analysis can be completely automated by means of computer aided identification of protein differences. For example, using an electronic detection system such as a laser scanning system with a photo multiplier tube or a charged-coupled device (CCD) camera and a white light source, multiple electronic images are made of the wet gel using different known filter sets to accommodate the different spectral characteristics of the labels. One image views fluorescence of the first dye using a first filter appropriate to filter out all light except that emitted at the wavelength of the first dye and a further image views fluorescence of a second dye using a second filter, appropriate to filter out all light except that emitted at the wavelength of the second dye etc. Proprietary filter combinations (often referred to as cubes or blocks) are commercially available that contain a combination of dichroic mirrors and filters capable of exciting fluorescent chromophores and diverting the resulting secondary fluorescence to the eyepieces or camera tube. Filtration is also used to reduce background fluorescence or inherent autofluorescence originating from either the sample itself or the gel.

Hence, in a preferred embodiment, capturing separate images of the labeled proteinaceous molecule comprises

(i) capturing a first image of the labeled proteinaceous molecule using a first filter or filters that only allow(s) the passage of light having the wavelength of the luminescent light emitted by a first dye within the set of dyes;
(ii) capturing at least one further image of the labeled proteinaceous molecule using another filter or filters that only allow(s) the passage of light having the wavelength of the luminescent light emitted by another dye within the set of dyes.

Preferably, contact between the glass cassette of the gel with the glass plate of the scanner is avoided to prevent the appearance of disturbing Newton rings. Newton rings are a type of interference pattern caused by light being reflected as it passes through multiple surfaces. This problem is especially common when transparent gels are scanned on a flatbed scanner where light must pass through a number of surfaces before being converted to digital information. In order to prevent the appearance of Newton rings, a film scanner especially for this purpose could be used, or film holders could be used to eliminate bending of the gel and to lift it from contact with the scanner's glass-plate surface. Alternatively, film mounting oil may be used to remove the air gap between the glass and the gel or the gel may be sandwiched between pieces of anti-Newton glass.

Linearity of a laser scanner is the signal range over which the instrument yields a linear response to fluorochrome concentration and is therefore useful for accurate quantitation. A scanner with a wide dynamic range can detect and accurately quantify signals from both very low-density and very high-intensity targets in the same scan. The linear dynamic range of most laser scanner instruments is between 10⁴ and 10⁵. In a preferred embodiment, the most intensive gel spot is examined with regard to its pixel intensity by means of an image analysis software package, the pixel factor not being allowed to exceed the value of 10⁵ in order to ensure linearity.

In a further embodiment, the method comprises normalizing the captured images to a common intensity range. For this purpose, an internal control sample is provided. The internal standard is composed of equal parts of all samples of one experiment and is usually labelled with the fluorescence dye having the lowest molecular weight. The same internal standard is run on all gels within the experimental series. This creates an intrinsic link between internal standard and samples in each gel, matching the internal standards between gels. Quantitative comparisons of samples between gels are made based on the relative change of sample to its in-gel internal standard. The presence of the same standard sample on every gel enables accurate normalization of the individual samples.

As described hereafter, normalizing the captured images to a common intensity range can also be achieved by providing each sample with two dyes chosen from a set of dyes, wherein one dye is used as internal standard. In this embodiment of the inventive method, each sample is provided with a different dye chosen from the set of dyes and is provided with an additional dye from the set of dyes which is provided to all samples (internal standard), and wherein each dye within the set of dyes emits luminescent light at a wavelength that is sufficiently different from the emitted luminescent light of the remaining dyes in the set of dyes to provide a different light signal.

Preferably, the normalization operations comprise processing the captured images with a computer. Suitable image analysis software package are commercially available. For example, software packages for two-dimensional (2-D) protein gel analysis generally feature specialized algorithms for spot-finding and analysis routines for gel-to-gel comparisons. Other important tools in these software packages may include data normalization; background correction; gel matching and grouping; and database input of analysis results.

The method according to the invention further comprises aligning the captured images of the labeled proteinaceous molecule by means of image processing. Hence, the present invention further relates to an image processing method for processing the captured images, and more particularly to an image processing method for “warping” the captured images in order to bring the images or portions thereof into registration with one another. Preferably, also the image processing operations comprise processing the captured images with a computer.

The term “registration” is often used with respect to printing processes to indicate the correct relation or exact superimposition between colors in color printing. In the present invention, the term “registration” refers to superimposition and alignment of features in the captured images where the features include a common pattern, or portions of a common pattern in each image, the pattern being identifiable by the naked eye and/or computer.

The term “image processing” is generally considered to mean the (computerized) manipulation of images or sets of images in order to facilitate the extraction of information, either by visual inspection or by automated measurement. Image processing is commonly used to enhance the usefulness of an image by changing the intensity, contrast, borders, size, placement, etc., of an image or features in the image. Image processing is also used to identify, locate and/or measure features represented within the image.

An image processing algorithm is a set of steps that may be automatically and rapidly applied to an image by a computer. Computers and digital electronics have brought about a multitude of ways in which images may be processed and manipulated. Numerous mathematical algorithms may be used, and image data may be processed on the level of individual pixels (in computer terms, a pixel is a basic picture element and is the smallest unit of visual information in an image). Image warping is a type of image processing which deals with geometric transformations to image features.

The term “warping”, as used herein, refers to a process of applying geometric corrections to modify the shape of features and to change their spatial relationships. Another term used for a warping process is rubber-sheeting because the warping process can be likened to stretching a rubber sheet wherein portions of one or more images are stretched or shrunk in order to bring the spots on all the images into registration with one another and still maintain relative positional relationships between the spots. This method may be used to rotate, translate, shift, stretch and/or shrink portions of images to bring spots in the images into registration with one another.

Preferably, software-based image analysis tools are used to align the captured images of the labeled proteinaceous molecule. These tools primarily analyze bio-markers by quantifying individual proteins, and showing the separation between one or more protein “spots” on a scanned image of a two-dimensional electrophoresis gel. Additionally, these tools match spots between gels of similar samples to show, for example, proteomic differences between early and advanced stages of an illness. Modern software packages include advanced features, such as image warping, to try to compensate for running differences between spots, and the generation of picking lists for the automated in-gel digestion of protein spots, leading to the identification of the proteins by mass spectrometry. Suitable software packages include Delta2D (Decodon), Dymension 3 (Syngene) and SameSpot (NonLinear Dynamic, Ltd.)—among others.

In a preferred embodiment, aligning the captured images of the labeled proteinaceous molecule comprises adjusting the position of the labeled proteinaceous molecule using image processing operations, whereupon the labeled proteinaceous molecule has a relative electrophoretic mobility that is the same as the electrophoretic mobility of the proteinaceous molecule labeled with another dye within the set of dyes. Such an alignment generally comprises stretching or shrunking portions of one or more images in order to bring the spots on all the images into registration with one another and still maintain relative positional relationships between the spots. Preferably, a fusion image is generated by the software package, which fusion picture represents all protein spots of the used gel images.

Although the step of aligning the captured images of the labeled proteinaceous molecule by means of image processing is highly desirable, it can also be omitted if there is no need of creating a printed fusion picture, where the spots on all the gel images are brought into registration with one another, the fusion picture thus representing all protein spots of the used gel images.

The method according to the present invention further comprises obtaining a value indicative for the differences in the amount of the labeled proteinaceous molecule within the plurality of samples. In a preferred embodiment of the inventive method, obtaining a value indicative for the differences in the amount of the labeled proteinaceous molecule within the plurality of samples comprises processing the aligned images of the labeled proteinaceous molecule to determine the difference in luminescent intensity. Each spot in a gel electrophoresis image may be composed of one or more pixels. Each pixel, or individual image element, has an associated intensity value. The intensity value represents the darkness of the image, and gives the pixel the capability to range from white to black. A proteinaceous molecule that is unique or of different relative concentration to one sample will have a different ratio of fluorescence intensity from the majority of protein spots, and will produce a color specific for one or the other of the samples, depending on the fluorescent dye used. Preferably, processing the aligned images of the labeled proteinaceous molecule comprises performing arithmetic operations on values representative of pixel intensities in the aligned images of the labeled proteinaceous molecule. For this purpose, pixel intensity can be analyzed by measuring the intensity of the region of interest with a suitable software package as described above and correlating the differences in luminescent intensity with the differences in the amount of the labeled proteinaceous molecule within the plurality of samples.

Following analysis of the gels using appropriate software, labelled protein spots can be picked and identified using standard procedures. Typically, the spots of interest are picked from the gel and the isolated protein spots are digested (“in-gel digestion”) with a suitable enzyme, for example, trypsin, Lys-C, and the peptide pattern determined by mass-spectrometry, for example electrospray, or MALDI-MS. The identity of individual protein components may also be determined using MS/MS peptide sequencing. In order to unambiguously identify labelled proteins (and thus interacting proteins), dye-labelled components may be purified from unlabelled components, prior to separation and/or MS analysis using a variety of well known methods, for example by the use of solid phase antibodies that bind to the dyes. The protein spots of interest can e.g. be picked by a Spot Picking Ettan™ Spot Handling Workstation.

In another embodiment of the inventive method, each sample is provided with two dyes chosen from a set of dyes, wherein one dye is used as internal standard (this may be the fluorescence dye having the lowest molecular weight). Hence, each sample is provided with a different dye chosen from the set of dyes (e.g., DyeM in sample A and DyeL in sample B, etc.) and is additionally provided with another dye from the set of dyes (e.g., DyeS in samples A and B, etc.) which is provided to all samples as an internal standard, wherein each dye within the set of dyes emits luminescent light at a wavelength that is sufficiently different from the emitted luminescent light of the remaining dyes in the set of dyes to provide a different light signal. The “copies” of the proteinaceous molecule (i.e. the individual molecules representing the proteinaceous molecule in the sample) comprised by the sample are thus contacted with two dyes chosen from a set of dyes, wherein one of these two dyes is used as internal standard. Hence, when using the above described “minimal dye labeling” approach, about half of the copies of the proteinaceous molecule comprised by the sample is statistically labeled with the dye representing the internal standard and the other half of the copies of the proteinaceous molecule comprised by the sample is labeled with the other dye (if the sample is provided with equal amounts of the dyes). As described above, it is also possible to provide the sample with different amounts or concentrations of the dyes; e.g., the sample is provided with a dye mixture, wherein the amount of the dye representing the internal standard is four times higher than the amount of the other dye.

Subsequently, the samples (each provided with a mixture of two fluorescent dyes) are combined and the combined samples are fractionated by means of electrophoresis (as described above). This embodiment has the advantage that there is no further need for the provision of an internal control sample which is composed of equal parts of all samples of one experiment and is run on all gels within the experimental series to allow normalizing the captured images. This embodiment thus relieves the practitioner of additionally labeling an internal control sample since each sample is already provided with an internal standard. Hence, this also avoids possible dilution errors when preparing an internal control sample which is composed of equal parts of all samples of one experiment.

This embodiment of the inventive method comprises:
(a) providing a plurality of samples, wherein at least one sample contains the proteinaceous molecule;
(b) contacting the proteinaceous molecule with two different dyes chosen from a set of dyes to label the proteinaceous molecule, wherein each sample is provided with a different dye chosen from the set of dyes and is provided with an additional dye from the set of dyes which is provided to all samples, and wherein each dye within the set of dyes emits luminescent light at a wavelength that is sufficiently different from the emitted luminescent light of the remaining dyes in the set of dyes to provide a different light signal;
(c) combining the samples;
(d) fractionating the combined samples by means of electrophoresis, wherein the labeled proteinaceous molecule has a relative electrophoretic mobility that differs from the electrophoretic mobility of the proteinaceous molecule labeled with another dye within the set of dyes;
(e) capturing separate images of the labeled proteinaceous molecule at the different wavelengths of emitted luminescence;
(f) aligning the captured images of the labeled proteinaceous molecule by means of image processing; and
(g) obtaining a value indicative for the differences in the amount of the labeled proteinaceous molecule within the plurality of samples.

In another embodiment of the inventive method, the plurality of samples comprises at least four (six) samples (e.g., samples A, B, C, D, etc.). The first two (three) samples are each provided with a different dye chosen from the set of dyes (e.g., DyeM in sample A, DyeL in sample B, etc.), and an internal control sample (e.g., sample IC1) is provided which is composed of equal parts of all samples of the first sample duo (trio) (e.g., equal parts of samples A, B, etc.). As described above, this internal control sample is provided with another dye chosen from the set of dyes (e.g., DyeS). It is understood that each dye within the set of dyes emits luminescent light at a wavelength that is sufficiently different from the emitted luminescent light of the remaining dyes in the set of dyes to provide a different light signal. The proteinaceous molecule is thus contacted with a dye chosen from a set of dyes to label the proteinaceous molecule, wherein each sample is provided with a different dye. Subsequently, equal parts of the first sample duo (trio) (e.g., DyeM in sample A, DyeL in sample B, etc.) and of the internal control sample (e.g., DyeS in sample IC1) are combined, are fractionated by means of electrophoresis (e.g., “gel 1”) and are processed as described above. Similarly, two (three) further samples are again each provided with a different dye chosen from the same set of dyes (e.g., DyeM in sample C, DyeL in sample D, etc.) and a further internal control sample (e.g., sample IC2) is provided which is composed of equal parts of all samples of the further sample duo (trio) (e.g., equal parts of samples C, D, etc.). As described above, this internal control sample is provided with another dye chosen from the same set of dyes (e.g., DyeS). Subsequently, equal parts of the further sample duo (trio) (e.g., DyeM in sample C, DyeL in sample D, etc.) and of the further internal control sample (e.g., DyeS in sample IC2) are combined, are fractionated by means of electrophoresis (e.g., “gel 2”) and are processed as described above. Accordingly, two (three) further samples can be processed as described above (e.g., “gel 3”, etc.). Furthermore, equal parts of all samples are also combined (e.g., DyeM in sample A, DyeL in sample B, DyeM in sample C, DyeL in sample D, DyeS in sample IC₁, DyeS in sample IC2, etc.) and the combined samples are fractionated by means of electrophoresis (i.e. “external standard gel”) and are processed as described above.

In this embodiment, by comparing the images of the gels (e.g., “gel 1”, “gel 2” and the “external standard gel”) in gel-to-gel comparisons, it is therefore possible to provide an external standard using each of the different fluorescent dyes as a standard. Suitable image analysis software package featuring analysis routines for gel-to-gel comparisons are commercially available. This embodiment has the advantage that correlating the differences in luminescent intensity with the differences in the amount of the labeled proteinaceous molecule within the plurality of samples is greatly improved since different standards and gel-to-gel comparisons provide for an exacter statistical analysis (see below).

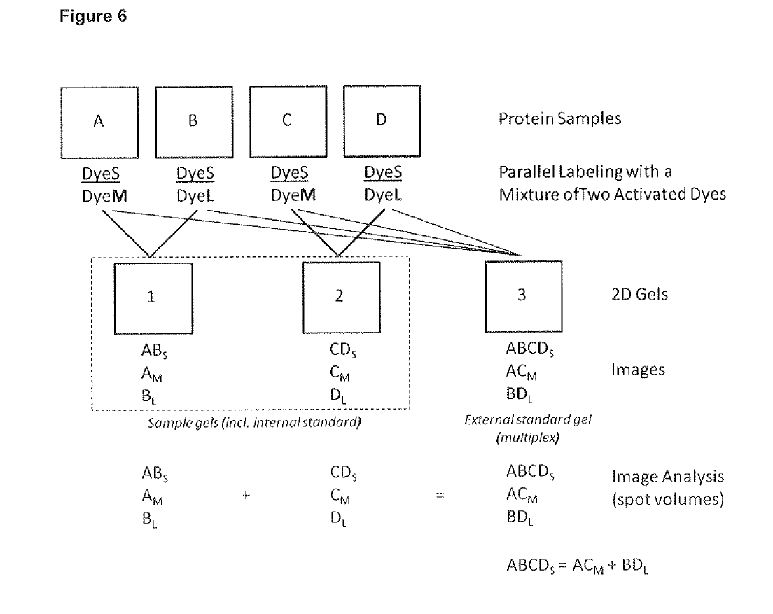

In yet another embodiment of the inventive method, the plurality of samples comprises at least four (six) samples (e.g., samples A, B, C, D, etc.). As can be gathered from FIG. 6, each sample is provided with two dyes chosen from a set of dyes, wherein one dye is used as internal standard (this may be the fluorescence dye having the lowest molecular weight). The first two (three) samples are each provided with a different dye chosen from the set of dyes (e.g., DyeM in sample A, DyeL in sample B, etc.) and are additionally provided with another dye chosen from the set of dyes (e.g., DyeS in samples A and B, etc.) which is provided to all samples as an internal standard. It is understood that each dye within the set of dyes emits luminescent light at a wavelength that is sufficiently different from the emitted luminescent light of the remaining dyes in the set of dyes to provide a different light signal. As explained above, the “copies” of the proteinaceous molecule (i.e. the individual molecules representing the proteinaceous molecule in the sample) comprised by the sample are thus contacted with two dyes chosen from a set of dyes, wherein one of these two dyes is used as internal standard. Subsequently, equal parts of the first sample duo (trio) (e.g., DyeM and DyeS in sample A, DyeL and DyeS in sample B, etc.) are combined, are fractionated by means of electrophoresis (e.g., “gel 1”) and are processed as described above. Similarly, two (three) further samples are each provided with a different dye chosen from the same set of dyes (e.g., DyeM in sample C, DyeL in sample D, etc.) and are additionally provided with another dye chosen from the same set of dyes (e.g., DyeS in samples C and D, etc.) which is provided to all samples as an internal standard. Subsequently, equal parts of the further sample duo (trio) (e.g., DyeM and DyeS in sample C, DyeL and DyeS in sample D, etc.) are combined and are fractionated by means of electrophoresis (e.g., “gel 2”). Accordingly, two (three) further samples can be processed as described above (e.g., “gel 3”, etc.). Furthermore, equal parts of all samples are also combined (e.g., DyeM and DyeS in sample A, DyeL and DyeS in sample B, DyeM and DyeS in sample C, DyeL and DyeS in sample D, etc.) and the combined samples are fractionated by means of electrophoresis (i.e. “external standard gel”) and are processed as described above.

In this embodiment, each electrophoresis gel thus includes an internal standard (e.g., DyeS). By comparing the internal standards of the gels (e.g., “gel 1”, “gel 2” and the “external standard gel”) in gel-to-gel comparisons, it is therefore possible to provide an external standard using each of the different fluorescent dyes as a standard. Suitable image analysis software package featuring analysis routines for gel-to-gel comparisons are commercially available. This embodiment has the advantage that correlating the differences in luminescent intensity with the differences in the amount of the labeled proteinaceous molecule within the plurality of samples is greatly improved since different standards and gel-to-gel comparisons provide for an exacter statistical analysis. For instance, a fusion picture of the images of “gel 1” and “gel 2” (using image analysis software) should be identical to the image of the “external standard gel” (cf. the image analysis as illustrated in FIG. 6).

In another embodiment, the present invention also relates to a kit for use in qualitatively and/or quantitatively determining a proteinaceous molecule in a plurality of samples by means of electrophoresis, wherein at least one sample contains the proteinaceous molecule, the kit comprising: a set of luminescent dyes, wherein each dye within the set of dyes emits luminescent light at a wavelength that is sufficiently different from the emitted luminescent light of the remaining dyes in the set of dyes to provide a different light signal, and wherein each dye within the set of dyes is configured so as to label the proteinaceous molecule, the labeled proteinaceous molecule having a relative electrophoretic mobility that differs from the electrophoretic mobility of the proteinaceous molecule labeled with another dye within the set of dyes.

Generally, each dye comprised by the kit is provided separately (freeze-dried or solved in a suitable organic solvent). However, the kit can also comprise a set of luminescent dyes being provided as mixtures of at least two dyes chosen from the set of dyes, wherein one dye (this may be the fluorescence dye having the lowest molecular weight) is used as internal standard and is thus included in each mixture, and wherein each mixture additionally includes at least another dye chosen from the set of dyes, the at least another dye preferably not being included in the other mixtures comprised by the kit. Each mixture can include the two (or more) dyes either in equal or different amounts or concentrations. The dyes may be provided freeze-dried or solved in a suitable organic solvent, such as DMF.

Preferably, the kit further comprises at least one electrophoresis gel or at least one electrophoresis gel set. The “electrophoresis gel set” comprises at least one of the following items: tank with cooling base and lid with power leads and connectors, tube gel module, one or more capillary glass tubes, one or more blanking stoppers, extraction platform, casting base unit, one or more notched glass plates, one or more plain glass plates, gel running module, one or more combs (sample slot+reference well), one or more spacer sets, pack of spacer aligners. Preferably, the electrophoresis gel set is designed to run 80 mm capillary tube gels, followed by vertical gels. The term “electrophoresis gels” denotes any precast polyacrylamide gels with performed wells and a stacking gel.

The kit may further comprise materials for quenching the labeling reaction. Alternatively or in combination, any suitable known quenching material may be used. Preferably, the kit comprises lysine for quenching the labeling reaction. The kit may also include buffers for running an electrophoresis.

In a preferred embodiment, the kit comprises a set of luminescent dyes including three fluorescent dyes (such as DyeS, DyeM, DyeL as described hereafter), a solvent (such as DMF) and a solution for quenching the labeling reaction comprising lysine, wherein the dyes may be provided freeze-dried or solved in the solvent provided in the kit (each in the same predetermined concentration). If the dyes are provided as a solution, the solutions may also comprise substances to improve the dyes' stability as well as storage and transport capabilities.

In a further preferred embodiment, the kit comprises a set of luminescent dyes including three fluorescent dyes (such as DyeS, DyeM, DyeL as described hereafter), DMF and a solution for quenching the labeling reaction comprising lysine, wherein the dyes are provided as a solution of DMF. Alternatively, any other suitable organic solvent may be used in the kit. In this embodiment, at least one dye is provided in a higher concentration than the other dyes. For example, one activated dye may be provided in an amount or a concentration that is two to five, preferably four times higher than the amount or the concentration of the other dyes (inter alia, DyeS as described hereafter is provided in a concentration of 10 nmol and DyeM and DyeL as described hereafter are each provided in a concentration of 2.5 nmol). This has the effect of increasing the spot intensity of the proteinaceous molecule labeled with the dye provided in a higher concentration, which may have an advantageous impact on image analysis. The solutions may comprise substances to improve the dyes' stability as well as storage and transport capabilities.

In a further preferred embodiment, the kit comprises a set of luminescent dyes including two fluorescent dyes (such as DyeS and DyeM or DyeS and DyeL as described hereafter), a solvent (such as DMF) and a solution for quenching the labeling reaction comprising lysine, wherein the dyes may be provided freeze-dried or solved in the solvent provided in the kit (each in the same predetermined concentration). If the dyes are provided as a solution, the solutions may also comprise substances to improve the dyes' stability as well as storage and transport capabilities. This kit may be used for the analysis of two samples.

In a further preferred embodiment, the kit comprises a set of luminescent dyes including four fluorescent dyes (such as DyeS, DyeM, DyeL as described hereafter and an additional fluorescent dye), a solvent (such as DMF) and a solution for quenching the labeling reaction comprising lysine, wherein the dyes may be provided freeze-dried or solved in the solvent provided in the kit (each in the same predetermined concentration). If the dyes are provided as a solution, the solutions may also comprise substances to improve the dyes' stability as well as storage and transport capabilities. This kit may be used for the analysis of four samples.

In another embodiment, the present invention also relates to the use of a set of luminescent dyes for the qualitative and/or quantitative determination of a proteinaceous molecule in a plurality of samples by means of electrophoresis, wherein each dye within the set of dyes emits luminescent light at a wavelength that is sufficiently different from the emitted luminescent light of the remaining dyes in the set of dyes to provide a different light signal, is configured so as to label the proteinaceous molecule, the labeled proteinaceous molecule having a relative electrophoretic mobility that differs from the electrophoretic mobility of the proteinaceous molecule labeled with another dye within the set of dyes.

Reproducible changes in protein expression, modifications, or interactions can be detected using the inventive method. Changes in protein expression in cells as well as bodily fluids can readily be identified and monitored by treating with natural agonists or chemical agents. Two-dimensional electrophoresis can be used to study differential expression of proteins in healthy and diseased tissues. Proteins that are more abundant in disease tissue may be considered as diagnostic disease markers or potential drug targets. Since a single two-dimensional electrophoresis can resolve thousands of proteins it can also be used for cell map proteomics as well as for characterizing subproteomes.

The method and the kit according to the invention can thus be used in the following fields of application:

a) as a biological tool for comparing the protein content of different samples in parallel;
b) as a biological tool for studying post-translational protein modifications in different samples in parallel;
c) as a diagnostic tool for comparing the protein content in patient samples with standard values and identifying significant deviations which are connected to a particular disease.

While the above invention has been described with respect to some of its preferred embodiments, this is in no way to limit the scope of the invention. The person skilled in the art is clearly aware of further embodiments and alterations to the previously described embodiments that are still within the scope of the present invention.

### EXAMPLES

### Protein Extraction

For the protein extraction, 0.8 g frozen and ground plant material (leaf or root) was used. The sample was provided with a threefold volume (2.4 ml) of an extraction buffer (100 mM HEPES-KOH (pH 7.5), 5% glycerol, 5 mM EDTA, 0.1% f3-mercaptoethanol, 1% proteinase inhibitor) and was vigorously shaken for 10 minutes at 4° C. The sample was then centrifuged for 10 minutes with 5000 rpm (Eppendorf centrifuge 5403) at 4° C. The supernatant was removed, the centrifugation step was repeated and the supernatant was then filtrated (Rotilabo® syringe filter 0.45 μm, Roth). The clear filtrate was provided with about the same volume of phenol. The solution was vigorously shaken and incubated on ice for 5 minutes. To obtain an optimal phase partition, the solution was centrifuged for 10 minutes with 5000 rpm at 4° C. The lower phase was transferred and provided with about the same volume of Re extraction buffer (100 mM Tris-HCl (pH 8.4), 20 mM KCl, 10 mM EDTA, 0.4% β-mercaptoethanol). After mixing the solution was centrifuged for 10 minutes with 5000 rpm at 4° C. The lower phase was transferred, provided with the same volume of Re extraction buffer and again centrifuged. After transferring the lower phase into a new vessel, the lower phase was provided with about the fivefold volume of precipitation solution (100 mM ammonium acetate in methanol, −20° C.). The solution was mixed and the proteins were precipitated overnight at −20° C.
The solution was centrifuged for 10 minutes with 5000 rpm at 4° C. and the supernatant was discarded. The formed pellet was initially washed with 3 ml precipitation solution, then twice with washing buffer (80% acetone, 20% Tris-HCl (pH 7.5)). The pellet was dried for about 10 minutes at room temperature and was dissolved in about 100 to 150 μl lysis buffer (30 mM Tris-HCl (pH 8.5), 7 M urea, 2 M thiourea, 4% CHAPS).

### Determination of the Protein Concentration

The amount of protein contained in the sample was determined using a 2D Quant Kit (GE Healthcare). The results were double-checked, each time using 1 and 2 μl of the sample, respectively. Additionally, a BSA straight calibration line having the concentrations 0, 5, 10, 15, 20 and 25 μg was set up. The extinction was measured by means of a photometer at a wavelength of 480 nm.

### Selection of Dyes

Three activated dyes in the form of NHS(N-hydroxysuccinimide) were provided to compose the exemplary set of dyes:

### (i) “DyeS”

(MW (g/mol): 549; λ_{abs} (nm): 495; εₘₐₓ (M⁻¹ cm⁻¹): 80.000)

### (ii) “DyeM”

(MW (g/mol): 791; λ_{abs} (nm): 554; εₘₐₓ (M⁻¹ cm⁻¹): 120.000)
(iii) “DyeL”
(MW (g/mol): 843; λ_{abs} (nm): 644; εₘₐₓ (M⁻¹ cm⁻¹): 150.000)
This exemplary set of dyes as well as other suitable sets of dyes are, inter alia, available from

### NH DyeAGNOSTICS UG

### Heinrich-Damerow-Str. 1

### 06120 Halle (Saale)

### Germany;

### (Commercial Register of Stendal HRB 9287);

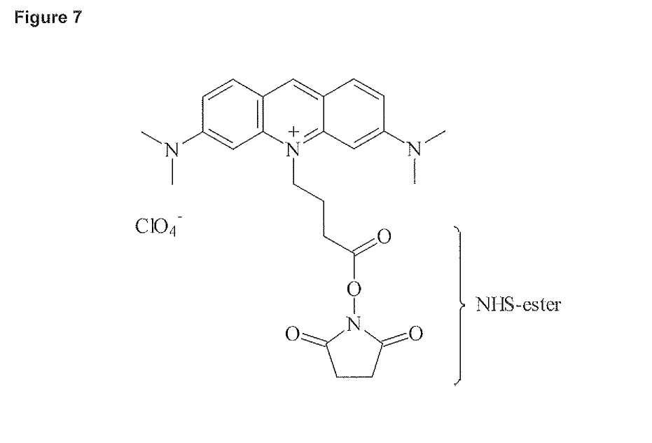

www.dyeagnostics.com.
NHS-esters represent the most commonly used amine-reactive reagents since they readily react with amino groups of proteins, i.e. the ε-amino groups of lysines or the amine terminus, forming a chemically stable amide bond between the dye and the protein. As can be gathered from FIG. 7, DyeS is an substituted acridine dye.

### Protein Labelling

When labelling the proteins the ratio of fluorescence dye-NHS ester to the amount of protein was selected so that there was an excess of protein. This approach ensured that each protein was labelled with only one dye molecule DyeS, DyeM or DyeL on a lysine side chain. Since for an optimal labelling reaction, the sample should have a pH of 8.5, the pH value was checked with unitest strips (Merck KgaA) prior to the reaction. For the labelling reaction a total of 50 μg protein was used each time, an adjustment to a concentration of 5 μg/μl being achieved by the addition of lysis buffer. The internal standard is composed of equal parts of all samples of one experiment and is usually labelled with the fluorescence dye DyeS. The labelling reaction was started by adding 400 pmol fluorescence dye NHS ester dissolved in 1 μl DMF. Thereafter the samples were incubated in the dark on ice for 30 minutes. The labelling reaction was interrupted by adding 1 μl of a 10 mM lysine solution (pH 9.0) and an incubation time of 10 minutes in the dark. The labelled protein samples were used for the preparation of a 2D multiplex fluorescence gel (24 cm IPG strips).

### Isoelectric Focussing (IEF)

Each fluorescence-labelled protein sample (V≈12 μl) was provided with about the same volume of 2× sample buffer (7 M urea, 2 M thiourea, 4% (w/v) CHAPS, 2% (v/v) IPG buffer, 130 mM DTT) and was incubated on ice for 10 minutes. Selecting the IPG buffer depends on the pH gradient of the used IPG strips. The samples were then combined resulting in a sample with a total protein amount of 150 μg. By adding 378 μl rehydration buffer (7 M urea, 2 M thiourea, 4% (w/v) CHAPS, 1% IPG buffer, 13 mM DTT), the total volume was adjusted to 450 μl. The solution was pipetted into a ceramic vessel and a 24 cm IPG strip (IPG DryStrip, GE Healthcare) was placed on it air bubble free. The IPG strip was then covered with 650 μl mineral oil (Immobiline™ DryStrip Cover Fluid, GE Healthcare). The IPG strips were actively rehydrated at 50 V per strip for 12 hours. The Ettan™ IPGPhor™ 3 IEF system (GE Healthcare) was used for focussing, a maximum amperage of 50 μA/Gel having been applied. After finishing focussing the focussed IPG strip was used straight away for the preparation of a 2D multiplex fluorescence gel or was stored at −80° C.

### SDS Gel Electrophoresis

### a) Equilibration

The focussed IPG strip was incubated for 15 minutes at room temperature with 2.5 ml equilibration solution A (6 M urea, 30% glycerine, 50 mM Tris-HCl (pH 8.8), 2% SDS, 0.002% (w/v) bromphenol blue, 1% DTT (freshly added)). Solution A was removed and the IPG strip was treated for 15 minutes at room temperature with 2.5 ml equilibration solution B (6 M urea, 30% glycerine, 50 mM Tris-HCl (pH 8.8), 2% SDS; 0.002% (w/v) bromphenol blue, 2.5% iodoacetimide (freshly added).

### b) Gel Electrophoresis

The equilibrated IPG strip was placed air bubble free onto the upper edge of a 12.5% SDS polyacrylamide gel and fixed with Agarose (0.5% SeaKam® LE Agarose, 100 ml SDS running buffer (25 mM Tris-HCl, 192 mM glycine, 0.2% (w/v) SDS)). To visualize the progress of the electrophoresis, 0.002% (w/v) bromphenol blue was added to the agarose. By means of SDS-PAGE according to Laemmli (Laemmli, 1970), the proteins were fractionated according to their size perpendicular to the first dimension. For this purpose an Ettan Dalttwelve device (GE Healthcare) was used (running buffer anode: 7.5 g SDS, 22.725 g TRIS, 7500 ml MiliQ water; 2× running buffer cathode: 6.06 g TRIS, 28.8 g glycine, 2 g SDS, 1000 ml MiliQ water). The 2D multiplex fluorescence gel was run at an electrical power of 1 Watt/Gel overnight.

### Digitalizing of the 2D Multiplex Fluorescence Gels

The gels were scanned using a Typhoon™ 9410 Variable Mode Imager (GE Healthcare), the gels remaining between the low fluorescent glass plates (GE Healthcare). During the scanning process the fluorescent dyes DyeS, DyeM and DyeL were successively excited with laser light with wavelengths of 488 nm, 532 nm and 633 nm. The emitted fluorescence was detected using emission filters 520 (band pass 40), 580 (band pass 30) and 670 nm (band pass 30). To prevent the appearance of disturbing Newton's Rings, contact between the glass cassette of the gel with the glass plate of the scanner was prevented by applying short strips of Kapton band or by using what are known as Gel Alignment Guides (GE Healthcare). A prescan of the gel at a low resolution of 1000 μm permitted fast optimization of the detection voltage for each fluorescent channel. To this end, the most intensive protein spot was examined with regard to its pixel intensity using the ImageQuant™ TL software, the pixel factor not being allowed to exceed the value of 100,000 in order to ensure linearity. The main scan of the 2D multiplex fluorescence gels was performed with a resolution of 100 μm.

### Image Analysis of the 2D Multiplex Fluorescence Gels

The Delta 2D software V3.4 (Decodon, Greifswald) was used to evaluate the experiments of producing 2D multiplex fluorescence gels. Each 2D multiplex fluorescence gel provided three pictures after scanning, each picture representing the protein pattern of the used protein sample. Because of the migration differences of identical but differently labelled proteins during SDS gel electrophoresis, these patterns were not exactly congruent to each other. By what is known as intra-gel warping, the position of the protein spots of the DyeM and DyeL dye channel was corrected with regard to the protein pattern of the DyeS dye gel (reference). Positional differences in the protein pattern between two or more gels were corrected by further inter-gel warping. An almost 100% congruence of identical protein spots in all dye channels and in all gels of a 2D multiplex fluorescence experiment could be achieved by means of this image processing.
After correction of the protein spots, a so-called fusion picture was generated by the software, which fusion picture represented all protein spots of the used gel pictures. On the basis of this fusion, spot detection was carried out and the position of the detected spots transferred to all gel pictures, whereby every gel picture possessed almost the same number of detected spots. This permitted the generation of complete expression profiles for the detected spots.

1. A method for qualitative and/or quantitative determination of a proteinaceous molecule, the method comprising:
(a) providing a plurality of samples, wherein at least one sample contains the proteinaceous molecule;
(b) contacting the proteinaceous molecule with a dye chosen from a set of dyes to label the proteinaceous molecule, wherein each sample is provided with a different dye, and wherein each dye within the set of dyes emits luminescent light at a wavelength that is sufficiently different from the emitted luminescent light of the remaining dyes in the set of dyes to provide a different light signal;
(c) combining the samples;
(d) fractionating the combined samples by means of electrophoresis, wherein the labeled proteinaceous molecule has a relative electrophoretic mobility that differs from the electrophoretic mobility of the proteinaceous molecule labeled with another dye within the set of dyes;
(e) capturing separate images of the labeled proteinaceous molecule at the different wavelengths of emitted luminescence;
(f) aligning the captured images of the labeled proteinaceous molecule by means of image processing; and
(g) obtaining a value indicative for the differences in the amount of the labeled proteinaceous molecule within the plurality of samples.

2. The method of claim 1, wherein the contacting step comprises
contacting the proteinaceous molecule with two different dyes chosen from the set of dyes to label the proteinaceous molecule, wherein each sample is provided with a different dye chosen from the set of dyes and is provided with an additional dye from the set of dyes which is provided to all samples, and wherein each dye within the set of dyes emits luminescent light at a wavelength that is sufficiently different from the emitted luminescent light of the remaining dyes in the set of dyes to provide a different light signal.

3. The method according to claim 1, wherein each sample of the plurality of samples contains the proteinaceous molecule.

4-5. (canceled)

6. The method according to claim 1, wherein the dyes are configured so as to covalently bind to at least one binding site of the proteinaceous molecule to label the proteinaceous molecule, the at least one binding site optionally being selected from the group consisting of amino, carboxy and sulfhydryl group, wherein the dyes optionally covalently bind to at least one lysine residue in the proteinaceous molecule.

7-9. (canceled)

10. The method according to claim 1, wherein each dye comprises a reactive group which is selected from the group consisting of isothiocyanate, maleimide and N-hydroxysuccinimide.

11. The method according to claim 1, wherein each dye within the set of dyes has a molecular weight that differs from the molecular weight of the remaining dyes in the set of dyes, wherein at least one dye within the set of dyes optionally has a molecular weight that differs from the molecular weight of the remaining dyes in the set of dyes by at least 100 g/mol, preferably at least 150 g/mol, more preferably at least 200 g/mol and most preferably at least 250 g/mol.

12. (canceled)

13. The method according to claim 1, wherein each dye has a net charge which will maintain the overall net charge of the proteinaceous molecule upon labeling the proteinaceous molecule, and/or wherein the labeled proteinaceous molecule has an overall net charge that does not substantially differ from the overall net charge of the unlabeled proteinaceous molecule, and/or wherein each dye within the set of dyes has a hydrophobicity that does not substantially differ from the hydrophobicity of the remaining dyes in the set of dyes.

14-15. (canceled)

16. The method according to claim 1, further comprising, prior to combining the samples, the step of quenching the labeling reaction.

17-18. (canceled)

19. The method according to claim 1, wherein capturing separate images of the labeled proteinaceous molecule comprises
(i) capturing a first image of the labeled proteinaceous molecule using a first filter or filters that only allow(s) the passage of light having the wavelength of the luminescent light emitted by a first dye within the set of dyes;
(ii) capturing at least one further image of the labeled proteinaceous molecule using another filter or filters that only allow(s) the passage of light having the wavelength of the luminescent light emitted by another dye within the set of dyes,
wherein the method optionally further comprises normalizing the captured images to a common intensity range.

20. (canceled)

21. The method according to claim 1, wherein aligning the captured images of the labeled proteinaceous molecule comprises adjusting the position of the labeled proteinaceous molecule using image processing operations, whereupon the labeled proteinaceous molecule has a relative electrophoretic mobility that is the same to the electrophoretic mobility of the proteinaceous molecule labeled with another dye within the set of dyes, wherein the image processing operations optionally comprise processing the captured images with a computer.

22. (canceled)

23. The method according to claim 1, wherein obtaining a value indicative for the differences in the amount of the labeled proteinaceous molecule within the plurality of samples comprises processing the aligned images of the labeled proteinaceous molecule to determine the difference in luminescent intensity, wherein processing the aligned images of the labeled proteinaceous molecule optionally comprises performing arithmetic operations on values representative of pixel intensities in the aligned images of the labeled proteinaceous molecule.

24. (canceled)

25. A kit for use in qualitatively and/or quantitatively determining a proteinaceous molecule in a plurality of samples by means of electrophoresis, wherein at least one sample contains the proteinaceous molecule, the kit comprising: a set of luminescent dyes, wherein each dye within the set of dyes emits luminescent light at a wavelength that is sufficiently different from the emitted luminescent light of the remaining dyes in the set of dyes to provide a different light signal, and wherein each dye within the set of dyes is configured so as to label the proteinaceous molecule, the labeled proteinaceous molecule having a relative electrophoretic mobility that differs from the electrophoretic mobility of the proteinaceous molecule labeled with another dye within the set of dyes, wherein each dye optionally comprises a reactive group which is selected from the group consisting of isothiocyanate, maleimide and N-hydroxysuccinimide.

26. The kit of claim 25, further comprising at least one electrophoresis gel or at least one electrophoresis gel set and/or materials for quenching the labeling reaction.

27-29. (canceled)

30. The kit according to claim 25, wherein at least one dye within the set of dyes has a molecular weight that differs from the molecular weight of the remaining dyes in the set of dyes by at least 100 g/mol, preferably at least 150 g/mol, more preferably at least 200 g/mol and most preferably at least 250 g/mol.

31. Use of a set of luminescent dyes for the qualitative and/or quantitative determination of a proteinaceous molecule in a plurality of samples by means of electrophoresis, wherein each dye within the set of dyes emits luminescent light at a wavelength that is sufficiently different from the emitted luminescent light of the remaining dyes in the set of dyes to provide a different light signal, is configured so as to label the proteinaceous molecule, the labeled proteinaceous molecule having a relative electrophoretic mobility that differs from the electrophoretic mobility of the proteinaceous molecule labeled with another dye within the set of dyes, wherein each dye optionally comprises a reactive group which is selected from the group consisting of isothiocyanate, maleimide and N-hydroxysuccinimide.

32-33. (canceled)

34. Use of claim 31, wherein at least one dye within the set of dyes has a molecular weight that differs from the molecular weight of the remaining dyes in the set of dyes by at least 100 g/mol, preferably at least 150 g/mol, more preferably at least 200 g/mol and most preferably at least 250 g/mol.
